SEQUENCE LISTING (6 sequences)

SEQ ID NO: 1 - DNA, 18 bp, Artificial Sequence
  18s Forward sequencing primer
aacccgttgaaccccatt

SEQ ID NO: 2 - DNA, 20 bp, Artificial Sequence
  18s Reverse sequencing primer
ccatccaatcggtagtagcg

SEQ ID NO: 3 - DNA, 20 bp, Artificial Sequence
  PROM2 Forward sequencing primer
gctcaggaacccaaacctgt

SEQ ID NO: 4 - DNA, 20 bp, Artificial Sequence
  PROM2 Reverse sequencing primer
ggcaggccatacatccttct

SEQ ID NO: 5 - DNA, 21 bp, Artificial Sequence
  PROM1 Forward sequencing primer
agtcggaaactggcagatagc

SEQ ID NO: 6 - DNA, 22 bp, Artificial Sequence
  PROM1 Reverse sequencing primer
ggtagtgttgtactgggccaat

(19) United States
(12) Patent Application Publication
(10) Pub. No.: US 2021/0380988 A1
(43) Pub. Date: Dec. 9, 2021

(54) Reducing Prominin2-Mediated Resistance to Ferroptotic Cell Death

(71) Applicant: University of Massachusetts, Boston, MA (US)

(72) Inventors: Arthur M. Mercurio, Boston, MA (US); Caitlin Westberg Brown, Northborough, MA (US)

(21) Appl. No.: 17/319,473

(22) Filed: May 13, 2021

Related U.S. Application Data
(60) Provisional application No. 63/091,132, filed on Oct. 13, 2020.
(60) Provisional application No. 63/024,125, filed on May 13, 2020.

(51) Int. Cl. C12N 15/113; A61K 31/437; A61K 31/496; A61K 31/15; A61P 35/00; A61K 31/4439; A61K 31/517; A61K 31/7088

(52) U.S. Cl. CPC C12N 15/1138; A61K 31/437; A61K 31/496; A61K 31/15; A61P 35/00; A61K 31/4439; C12N 2310/14; C12N 15/113; C12N 15/1137; A61K 31/7088; C12N 2310/321; C12N 2310/322; C12N 2320/31; A61K 31/517

(57) ABSTRACT

Compositions and methods for inducing tumor cell death by inhibiting prominin2 (PROM2) or heat shock factor protein 1 (HSF1), optionally in combination with an agent that promotes ferroptosis, preferably an inhibitor of glutathione peroxidase 4 (GPX4), e.g., to trigger ferroptosis in the tumor cells.

### CLAIM OF PRIORITY

This application claims the benefit of U.S. Provisional Patent Application Ser. Nos. 63/024,125, filed on May 13, 2020, and 63/091,132, filed on Oct. 13, 2020. The entire contents of the foregoing are hereby incorporated by reference.

### FEDERALLY SPONSORED RESEARCH OR DEVELOPMENT

This invention was made with Government support under Grant No. CA218085 awarded by the National Institutes of Health and Grant No. W81XWH-17-1-009 awarded by the Department of Defense. The Government has certain rights in the invention.

### SEQUENCE LISTING

The instant application contains a Sequence Listing which has been submitted electronically in ASCII format and is hereby incorporated by reference in its entirety. Said ASCII copy, created on Jun. 24, 2021, is named UMMS20-56_SL.txt and is 1,559 bytes in size.

### TECHNICAL FIELD

Provided herein are compositions and methods for inducing tumor cell death by inhibiting prominin2 (PROM2) or heat shock factor protein 1 (HSF1), optionally in combination with an agent that promotes ferroptosis, preferably an inhibitor of glutathione peroxidase 4 (GPX4), e.g., to overcome resistance to ferroptosis and ferroptosis-inducing agents in the tumor cells.

### BACKGROUND

Ferroptosis is a regulated form of non-apoptotic cell death characterized by the iron-dependent accumulation of lethal lipid reactive oxygen species (ROS) (Dixon et al., 2012; Yang et al., 2016; Yang and Stockwell, 2016). This process can contribute to pathological cell death in brain, kidney, heart and other tissues (Do Van et al., 2016; Fang et al., 2019; Friedmann Angeli et al., 2014; Hangauer M J, 2017; Hirschhorn and Stockwell, 2019). There is also interest in understanding the role of ferroptosis in cancer, and potentially exploiting this mechanism therapeutically (Dixon, 2019). Interestingly, ferroptosis sensitivity varies substantially between between cancer cells from distinct tissue lineages that express certain tumor suppressors, or have acquired unique cell drug-tolerant or de-differentiated cell states (Hangauer M J, 2017; Jiang et al., 2015; Tsoi et al., 2018; Viswanathan V S, 2017; Yang et al., 2014; Zhang et al., 2019). At the molecular level, mechanisms that can account for the observed variation in ferroptosis sensitivity between cells remain largely elusive.

Ferroptosis can be initiated by a number of stimuli that disrupt intracellular gluthione-mediated antioxidant systems or directly overload the cell with iron (Stockwell et al., 2017). For example, it is possible to induce ferroptosis by depriving cells of the thiol-containing amino acid cystine (the disulfide of cysteine), a key glutathione precursor, or by directly inhibiting the the reduced glutathione (GSH)-dependent phospholipid hydroperoxiodase glutathione peroxidase 4 (GPX4) (Dixon et al., 2012; Yang et al., 2014). GPX4 catalyzes the reduction of reactive lipid hydroperoxides to non-reactive lipid alcohols. This reaction is essential to prevent a buildup of lipid hydroperoxides, which when free iron is present can participate in Fenton chemistry reactions that lead to the generation of toxic lipid alkoxy radicals and subsequently other reactive lipid breakdown products (Dixon, 2019). The execution of ferroptosis therefore relies on the combined presence of oxidizable lipids (e.g. polyunsaturated phospholipids) and free iron which, in the absence of sufficient GPX4-mediated lipid peroxide detoxification, result in oxidative destruction of the plasma membrane and other internal organelle membranes (Agmon et al., 2018; Magtanong et al., 2019). Physiological stress such as detachment of cells from the extracellular matrix can increase ROS and is sufficient to trigger ferroptosis if GPX4 activity is inhibited (Brown C W, 2017).

Iron is essential for cell growth but can also promote toxic ROS formation, as occurs during ferroptosis. How intracellular iron homeostasis contributes to ferroptosis sensitivity is only partially understood. Iron can be taken into cells via the transferrin/transferrin receptor system, and genetic silencing of transferrin receptor partially inhibits ferroptosis (Gao et al., 2016; Yang and Stockwell, 2008). NCOA4-mediated, lysosomal-dependent degradation of the iron storage protein ferritin contributes to the pool of free intracellular iron and thereby promotes ferroptosis; genetic depletion of NCOA4 or chemical inhibition of lysosomal acidification both attenuate ferroptosis (Gao et al., 2016; Hou et al., 2016; Torii et al., 2016). Furthermore, manipulation of ferroportin-mediated iron export can alter ferroptosis sensitivity, with reduced ferroportin expression leading to less iron export and increased ferroptosis sensitivity (Geng N, 2018; Ma et al., 2016). Thus, the modulation of intracellular free iron levels by the cellular iron homeostatic network emerges as a key regulator of ferroptosis sensitivity. Interestingly, intracellular free iron levels can vary between cancer cells and cancer cell states (e.g. in metastatic versus non-metastatic cells), which might be expected to contribute to differences in ferroptosis sensitivity (Geng N, 2018). There is also some evidence that iron levels may be dynamically regulated within the cell in response to ferroptosis-inducing conditions, suggesting that uncharacterized regulatory mechanisms may alter intracellular iron, possibly in an attempt to negatively regulate the onset of ferroptosis (Ma et al., 2016).

### SUMMARY

Described herein are experiments that used an unbiased approach to investigate how mammary epithelial and breast carcinoma cells survive in response to pharmacological and physiological ferroptotic stress. As shown herein, cells can resist the onset of ferroptosis by dynamically upregulating an unprecedented iron export pathway involving multi-vesicular body (MVB)/exosome trafficking of ferritin and iron out of the cell. This process limits the intracellular accumulation of free iron and inhibits the onset of ferroptosis. By contrast, inactivation of this program, either normally or following chemical or genetic inhibition of key steps of this process, substantially increases ferroptosis sensitivity. These findings pinpoint a mechanism that leads to variations in ferroptosis sensitivity and.

Thus, provided herein are methods of treating a subject who has cancer. The methods include administering to the subject an effective amount of (i) an inhibitor of prominin 2 (PROM2); or (ii) an effective amount of an inhibitor of heat shock factor protein 1 (HSF1) and an effective amount of one or more agents that promote ferroptosis, preferably an inhibitor of glutathione peroxidase 4 (GPX4).

In some embodiments, the inhibitor of PROM2 or the inhibitor of HSF1 is an inhibitory nucleic acid, e.g., an antisense oligonucleotide (ASO), small interfering RNA (siRNA), or small hairpin RNA (shRNA) that binds to a PROM2 or HSF1 nucleic acid. In some embodiments, the inhibitory nucleic acid comprises one or more modified bonds or bases. In some embodiments, the modified bases comprise at least one nucleotide modified at the 2′ position of the sugar, e.g., a 2′-O-alkyl, 2′-O-alkyl-O-alkyl or 2′-fluoro-modified nucleotide. In some embodiments, the modified bonds comprise modified backbone bonds, e.g., the modified backbone bonds comprises phosphorothioates, phosphotriesters, methyl phosphonates, short chain alkyl or cycloalkyl intersugar linkages or short chain heteroatomic or heterocyclic intersugar linkages.

In some embodiments, the methods include administering one or more agents that promote ferroptosis, preferably an inhibitor of glutathione peroxidase 4 (GPX4), e.g., RSL3, Erastin, Imidazole ketone erastin (IKE), ML210, FIN56, Auranofin, Sulfasalazine, Artesunate, Artemisinin, Dihydroartemisinin, Sorafenib, Buthionine sulfoximine (BSO), Altretamine, Almitrine, and ML162.

In some embodiments, the agent that promotes ferroptosis is ascorbic acid; ultrasmall nanoparticles; elastin or carbonyl erastin analogs that inhibit GPX4; or quinazolinone inhibitors of GPX4.

In some embodiments, the agent that promotes ferroptosis is an inhibitory nucleic acid molecule that targets GPX4, e.g., an antisense oligonucleotide (ASO), small interfering RNA (siRNA), or small hairpin RNA (shRNA) that binds to a GPX4 nucleic acid. In some embodiments, the inhibitory nucleic acid comprises one or more modified bonds or bases. In some embodiments, the modified bases comprise at least one nucleotide modified at the 2′ position of the sugar, e.g., a 2′-O-alkyl, 2′-O-alkyl-O-alkyl or 2′-fluoro-modified nucleotide. In some embodiments, the modified bonds comprise modified backbone bonds, e.g., the modified backbone bonds comprises phosphorothioates, phosphotriesters, methyl phosphonates, short chain alkyl or cycloalkyl intersugar linkages or short chain heteroatomic or heterocyclic intersugar linkages.

In some embodiments, the inhibitor of HSF1 is KRIBB11 n(N(2)-(1H-indazole-5-yl)-N(6)-methyl-3-nitropyridine-2,6-diamine); Quercetin; Stresgenin B; Triplotide; Cantharidin; Fisetin; Rocaglamide A; 2,4-Bis(4-hydroxy benzyl)Phenol; CL-43; celasterol; cantharidin; 2,4-Bis(4-hydroxybenzyl)phenol; minnelide; KNK437; NZ-28; PW3405; IHSF115; 4,6-Disubstituted Pyrimidine; CCT361814; α-Acyl amino carboxamides; emunin; or bisamide (CCT251236).

Also provided herein are compositions comprising:

(i) an inhibitory nucleic acid targeting prominin 2 (PROM2) or heat shock factor protein 1 (HSF1); and (ii) an agent that promotes ferroptosis, preferably an inhibitor of glutathione peroxidase 4 (GPX4).

In some embodiments, the agent that promotes ferroptosis is selected from the group consisting of RSL3, Erastin, Imidazole ketone erastin (IKE), ML210, FIN56, Auranofin, Sulfasalazine, Artesunate, Artemisinin, Dihydroartemisinin, Sorafenib, Buthionine sulfoximine (BSO), Altretamine, Almitrine, and ML162.

In some embodiments, the agent that promotes ferroptosis is ascorbic acid; ultrasmall nanoparticles; elastin or carbonyl erastin analogs that inhibit GPX4; quinazolinone inhibitors of GPX4, or an inhibitory nucleic acid molecule that targets GPX4.

In some embodiments, the inhibitory nucleic acid targeting PROM2, HSF1, or GPX4 is an antisense oligonucleotide (ASO), small interfering RNA (siRNA), or small hairpin RNA (shRNA) that binds to a PROM2, HSF1, or GPX4 nucleic acid.

In some embodiments, the inhibitory nucleic acid comprises one or more modified bonds or bases. In some embodiments, the modified bases comprise at least one nucleotide modified at the 2′ position of the sugar, e.g., a 2′-O-alkyl, 2′-O-alkyl-O-alkyl or 2′-fluoro-modified nucleotide. In some embodiments, the modified bonds comprise modified backbone bonds, e.g., the modified backbone bonds comprises phosphorothioates, phosphotriesters, methyl phosphonates, short chain alkyl or cycloalkyl intersugar linkages or short chain heteroatomic or heterocyclic intersugar linkages.

In some embodiments, the inhibitor of HSF1 is KRIBB11 n(N(2)-(1H-indazole-5-yl)-N(6)-methyl-3-nitropyridine-2,6-diamine); Quercetin; Stresgenin B; Triplotide; Cantharidin; Fisetin; Rocaglamide A; 2,4-Bis(4-hydroxy benzyl)Phenol; CL-43; celasterol; cantharidin; 2,4-Bis(4-hydroxybenzyl)phenol; minnelide; KNK437; NZ-28; PW3405; IHSF115; 4,6-Disubstituted Pyrimidine; CCT361814; α-Acyl amino carboxamides; emunin; or bisamide (CCT251236).

In some embodiments, the composition further includes a pharmaceutically acceptable carrier.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F. Prominin2 is a ferroptosis stress response protein. (A) MCF10A cells were detached from ECM for 30 min, 1 hr, 2 hrs or 3 hrs. Extracts from these cells were immunoblotted for prominin2 and actin. Immunoblot is representative of three independent experiments. (B) MCF10A and Hs578t cells were transfected with either control siRNA or prominin2 siRNA for 48 hrs and then maintained under adherent or ECM-detached conditions for 2 hrs. PROM2 mRNA expression was quantified by qPCR. Shown is representative experiment of three independent replicates. (C) siControl or siProminin2-treated MCF10A and Hs578t cells were maintained in ECM detached conditions for 24 hrs in the presence of either DMSO, ferrostatin-1 (2 μM), ZVAD-fmk (25 μM) or ferrostatin-1 and ZVAD-fmk and the number of viable cells was quantified. Shown is representative experiment of three independent replicates. (D) Adherent MCF10A, Hs578t and MDA-MB-231 cells were treated with either DMSO or RSL3 for 2 hrs and PROM2 mRNA expression was quantified by qPCR. Shown is representative experiment of three independent replicates. (E) Extracts from the cells in (D) were immunoblotted with antibodies specific for prominin2 and tubulin. The densitometric analysis of these immunoblots (normalized to 1.0 for each cell line) obtained from analysis of three independent experiments in shown below the blots. (F) Adherent MCF10A, Hs578t and MDA-MB-231 cells were treated with DMSO or RSL3 24 hrs and the percent of surviving cells was quantified. Absorbance was normalized to DMSO control. Shown is representative experiment of three independent replicates.

FIGS. 2A-D. Prominin2 increases resistance to ferroptosis. (A) Prominin2 expression was diminished in adherent MCF10A cells for 48 hours using siRNA prior to treatment with RSL3, ML210, FIN56, or erastin for for 24 hrs. The percent of surviving cells was quantified and compared to cells transfected with an siRNA control. Absorbance was normalized to DMSO control. Shown is representative experiment of three independent replicates. See the IC₅₀ values for these conditions. (B) Prominin2 expression was diminished in adherent Hs578t cells for 48 hours using siRNA prior to treatment with RSL3, ML210, FIN56, or erastin for 24 hrs. The percent of surviving cells was quantified and compared to cells transfected with an siRNA control. Absorbance was normalized to DMSO control. Shown is representative experiment of three independent replicates. See the IC₅₀ values for these conditions. (C) Prominin2 expression was diminished in adherent MCF10A and Hs578t cells for 48 hours using siRNA. Cells were treated with either DMSO, RSL3 (5 μM) or RSL3 and ferrostatin-1 (2 μM) for 24 hrs and the percent of surviving cells was quantified. Shown is representative experiment of three independent replicates. (D) MDA-MB-231 cells were transfected with either a prominin2 expression construct or a vector control prior to treatment with either DMSO or RSL3 for 2 hrs. The percent of surviving cells was quantified. Absorbance was normalized to DMSO control. Shown is a representative experiment of three independent replicates.

FIGS. 3A-F. Prominin2 promotes the formation of multivesicular bodies (MVBs) in response to GPX4 inhibition. (A) Adherent MCF10A and Hs578t cells were treated with either DMSO or RSL3 for 2 hrs and immunostained using Abs specific for TSG101 and prominin2. Scale bar=10 μm. Also, for all experiments, image acquisition settings were the same for DMSO and RSL3-treated cells. Shown is representative image of three independent replicates. (B) Adherent Hs578t cells were treated with either DMSO or RSL3 for either 1, 1.5 or 2 hrs and immunostained using Abs specific for TSG101 and prominin2. Scale bar=10 μm. Shown is representative image of three independent replicates. (C) MCF10A cells were transfected with either control siRNA or prominin2 siRNA for 48 hrs and then treated with either DMSO or RSL3 for 1.5 hrs. These cells were fixed and processed for transmission electron microscopy (TEM). The frequency of MVBs (white box) per section was quantified (right graph). Shown is representative image of five total cells quantified per group. (D) MCF10A cells and were transfected with either control siRNA or prominin2 siRNA for 48 hrs and then treated with either DMSO or RSL3 for 2 hrs. These cells were then immunostained using Abs specific for TSG101 and prominin2. Scale bar=10 μm. Shown is representative image of three independent replicates. (E) Hs578t cells and were transfected with either control siRNA or prominin2 siRNA for 48 hrs and then treated with either DMSO or RSL3 for 2 hrs. These cells were then immunostained using Abs specific for TSG101 and prominin2. Scale bar=10 μm. Shown is representative image of three independent replicates. (F) MDA-MB-231 cells were transfected with either a prominin2 expression construct or a vector control prior to treatment with either DMSO or RSL3 for 2 hrs. These cells were then immunostained using antibodies specific for TSG101 and prominin2. Scale bar=10 μm. Shown is representative image of three independent replicates.

FIGS. 4A-F. Multivesicular body formation is required for evasion of ferroptosis. (A) TSG101 expression was diminished in adherent MCF10A cells for 48 hours using siRNA prior to treatment with DMSO or RSL3 for 24 hrs. The percent of surviving cells was quantified and compared to cells transfected with an siRNA control. Absorbance was normalized to DMSO control. Shown is representative experiment of three independent replicates. (B) TSG101 expression was diminished in adherent Hs578t cells for 48 hours using siRNA prior to treatment with DMSO or RSL3 for 24 hrs. The percent of surviving cells was quantified and compared to cells transfected with an siRNA control. Absorbance was normalized to DMSO control. Shown is representative experiment of three independent replicates. (C) TSG101 expression was diminished in adherent MCF10A cells for 48 hours using siRNA prior to treatment with DMSO, RSL3 or RSL3 and Ferrostatin-1 for 24 hrs. The percent of surviving cells was quantified and compared to cells transfected with an siRNA control. Absorbance was normalized to DMSO control. Shown is representative experiment of three independent replicates. (D) Exosomes were isolated from cell culture medium of MCF10A and Hs578t cells treated for 24 hours with 1 μM RSL3 by ultracentrifugation. The exosome pellets were lysed and protein concentration was assessed. Exosomes from RSL3 treated MCF10A and Hs578t cells were immunoblotted for TSG101, prominin2, and Alix. See TEM of exosomes. Shown is representative immunoblot of three independent collections. (E) Exosomes were isolated from cell culture medium of MCF10A and Hs578t cells treated for 24 hours with DMSO or 1 μM RSL3 by ultracentrifugation. The exosome pellets were lysed and protein concentration was assessed. Whole cell lysate and exosomes from MCF10A and Hs578t cells were immunoblotted for GM130. (F) MCF10A and Hs578t cells were treated with either DMSO, RSL3 (5 μM) or RSL3 and the exosome inhibitor GW4869 (20 μM) for 24 hrs and the percent of surviving cells was quantified. Shown is representative experiment of three independent replicates.

FIGS. 5A-F. Prominin2 and ferritin contribute to the regulation of free iron. (A) Adherent MCF10A and Hs578t cells were treated with either DMSO or RSL3 for 2 hrs and immunostained using Abs specific for ferritin and prominin2. Scale bar=10 μm. Images are representative of three independent experiments with similar results. (B) Exosomes purified from RSL3-treated MCF10A cells as described in FIG. 3F were immunoblotted for ferritin. (C) MDA-MB-231 cells were transfected with either a prominin2 expression construct or a vector control prior to treatment with either DMSO or 5 uM RSL3 for 2 hrs. These cells were then immunostained using antibodies specific for ferritin heavy chain and prominin2. Scale bar=10 μm. Shown are representative images of three independent experiments. (D) MCF10A cells were transfected with either control siRNA or ferritin heavy chain siRNA for 48 hrs and then treated with increasing concentrations of RSL3 for 24 hrs, and the percent of surviving cells was quantified. Shown is representative experiment of three independent replicates. (E) Hs578t cells were transfected with either control siRNA or ferritin heavy chain siRNA for 48 hrs and then treated with increasing concentrations of RSL3 for 24 hrs, and the percent of surviving cells was quantified. Shown is representative experiment of three independent replicates. (F) MCF10A and Hs578t cells were transfected with either control siRNA or ferritin siRNA for 48 hrs and then treated with increasing concentrations of either DMSO, RSL3 and ferrostatin-1, or RSL3 and 100 uM deferoxamine (DFO) for 24 hrs. The percent of surviving cells was then quantified. Shown is a representative experiment of three independent replicates.

FIGS. 6A-D. GPX4 inhibition alters free iron concentration. (A) Adherent MCF10A and Hs578t cells were transfected with either control siRNA or prominin2 siRNA for 48 hrs and then treated with 5 μM RSL3 for 2 hrs. Total free iron was quantified and reported in nM. Shown is a representative experiment of three independent replicates. (B) Adherent MCF10A and Hs578t cells were transfected with either control siRNA or ferritin heavy chain siRNA for 48 hrs and then treated with RSL3 for 2 hrs. Total free iron was quantified and reported in nM. Shown is a representative experiment of three independent replicates. (C) Adherent MDA-MB-231 cells were transfected with either a prominin2 expression construct or a vector control prior to treatment with either DMSO or RSL3 for 2 hrs. Total free iron was quantified and reported as nMoles. Shown is representative experiment of three independent replicates. (D) Adherent MCF10A and Hs578t cells were transfected with either control siRNA or TSG101 siRNA for 48 hrs and then treated with either DMSO or RSL3 for 2 hrs. Total free iron was quantified and reported as nanomoles. Shown is representative experiment of three independent replicates.

FIGS. 7A-D. Detachment from the extracellular matrix induces prominin2-dependent MVB formation to rescue ferroptosis. (A) Control and prominin2-depleted MCF10A cells were maintained in ECM-detached conditions for 24 hrs in the presence of either DMSO or deferoxamine and the number of viable cells was quantified. Shown is representative experiment of three independent replicates. (B) Control and prominin2-depleted MCF10A cells were maintained in ECM-detached conditions for the indicated times and total free iron was quantified. Shown is representative experiment of three independent replicates. (C) Control and prominin2-depleted MCF10A cells were maintained in ECM-detached conditions for 2 hrs and processed for TEM. The frequency of MVBs (white box) per section was quantified (right graph). Shown is representative image of five total cells quantified per group. (D) MCF10A and Hs578t cells were maintained in ECM-detached conditions for 2 hrs, fixed and immunostained for TSG101 and prominin2. Scale bar=10 μm. Shown are representative images of three independent experiments.

FIGS. 8A-B. (A) MCF10A and Hs578t cells were maintained in DMSO, 10 uM erastin, 250 μM H₂O₂, or 5 μM RSL3 for 2 hrs. Extracts from these cells were evaluated by qPCR for PROM1 and PROM2 expression. (B) MCF10A and Hs578t cells were transfected with 1 μg of prominin2 siRNA for 48 hours and extracts were immunoblotted for prominin2 and tubulin.

FIGS. 9A-D. (A) MCF10A control and prominin2-depleted cells were treated with RSL3, FIN56, ML210 or erastin as described in FIG. 2A. Data were analyzed for log IC₅₀ using Graphpad Prism software and four point logistic regression. Shown are representative experiments of three independent replicates. (B) Hs578t control and prominin2-depleted cells were treated with RSL3, FIN56, ML210, or erastin as described in FIG. 1H. Data was analyzed for Log IC₅₀ using Graphpad Prism software. Shown are representative experiments of three independent replicates. (C) MDA-MB-231 cells were transfected with a pCMV empty vector or pCMV-prominin2 plasmid and stably selected. Prominin2 expression was quantified by qPCR. (D) MDA-MB-231 vector and prominin2-expressing cells were treated with RSL3, FIN56, ML210 or erastin as described in FIG. 2A. Data was analyzed for Log IC₅₀ using Graphpad Prism software. Shown are representative experiments of three independent replicates.

FIGS. 10A-D. (A) Adherent MCF10A and Hs578t cells were treated with either DMSO or RSL3 for 2 hrs and immunostained using Abs specific for TSG101 and prominin2. Scale bar=10 μm. For all experiments, image acquisition settings were the same for DMSO and RSL3-treated cells. Shown is representative image of three independent replicates. (B) MCF10A cells were treated with either DMSO or RSL3 for 2 hrs and immunostained using Abs specific for prominin2 and either catalase, phospho-caveolin1, LC3, mitotracker or Rab5A. Scale bar=10 μm. (C) MCF10A and Hs578t cells were treated with either DMSO or RSL3 for 2 hrs and immunostained using Abs specific for Lamp1 and prominin2 Scale bar=10 μm. (D) Hs578t cells were pre-treated with either H₂O (control) or 20 mM NH₄Cl for 60 min. Cells were then washed and treated with either H₂O (control) or NH₄Cl, and RSL3 at the concentrations indicated for 24 hrs and the percent of surviving cells was quantified. Absorbance was normalized to DMSO control.

FIGS. 11A-C. (A) Adherent Hs578t cells were treated with either DMSO or RSL3 for either 1, 1.5 or 2 hrs and immunostained using Abs specific for TSG101 and prominin2. Scale bar=10 μm. Shown is representative image of three independent replicates. (B) Adherent MCF10A and Hs578t cells were treated with either DMSO or RSL3 for 2 hrs and immunostained using Abs specific for Alix and prominin2. Scale bar=10 μm. For all experiments, image acquisition settings were the same for DMSO and RSL3-treated cells. Shown is representative image of three independent replicates. (C) MCF10A and Hs578t cells were treated with either DMSO, RSL3 (5 μM) or RSL3 and the exosome inhibitor GW4869 (20 μM) for 24 hrs and the percent of surviving cells was quantified. Shown is representative experiment of three independent replicates.

FIGS. 12A-C. (A) MCF10A cells were transfected with either control siRNA or prominin2 siRNA for 48 hrs and then treated with either DMSO or RSL3 for 2 hrs. These cells were then immunostained using Abs specific for TSG101 and prominin2. Scale bar=10 μm. Shown is representative image of three independent replicates. (B) Hs578t cells were transfected with either control siRNA or prominin2 siRNA for 48 hrs and then treated with either DMSO or RSL3 for 2 hrs. These cells were then immunostained using Abs specific for TSG101 and prominin2. Scale bar=10 μm. Shown is representative image of three independent replicates. (C) MDA-MB-231 cells were transfected with either a prominin2 expression construct or a vector control prior to treatment with either DMSO or RSL3 for 2 hrs. These cells were then immunostained using antibodies specific for TSG101 and prominin2. Scale bar=10 μm. Shown is representative image of three independent replicates.

FIGS. 13A-D. (A) MCF10A and Hs578t cells were transfected with either control or TSG101 siRNA for 48 hours and extracts were immunoblotted for TSG101 and tubulin. (B) Exosomes isolated from MCF10A and Hs578t cell culture media were isolated as described in FIG. 4D, fixed and imaged by TEM. Exosomes are highlighted in black boxes. (C) MCF10A cells were treated with either DMSO or RSL3 for 2 hrs and immunostained using Abs specific for SLC40A1/ferroportin and prominin2 Scale bar=10 μm. (D) MCF10A and Hs578t cells were transfected with either control or ferritin light and heavy chain siRNA for 48 hours and extracts were immunoblotted for FTH1 and tubulin.

FIG. 14. Prominin2 expression is correlated with ferroptosis resistance. Analysis of the Broad Institute CTRP resource indicated that high levels of prominin2 expression are significantly correlated with resistance to the GPX4 inhibitor ML210 in cancer cell lines.

FIGS. 15A-G. 4HNE activates HSF1 to induce prominin2 expression. A) HSF1 was identified as a strong occupier of the PROM2 promoter B) MCF10A cells were treated with 25 uM 4HNE or 5 uM RSL3 for a range of exposure times from 0 to 120 minutes. mRNA was isolated from each time point and HSF 1 expression was quantified by qPCR. Shown is an average of three independent experiments with standard deviation. C) MCF10A cells were pretreated for 15 minutes with DMSO or 10 uM BIRB, followed by 60 minutes in DMSO, 25 uM 4HNE, 5 uM RSL3, 4HNE and BIRB, or RSL3 and BIRB. mRNA was isolated and HSF 1 expression was quantified by qPCR. Shown are three independent experiments with standard deviation. D) MCF10A cells were pretreated for 15 minutes with DMSO or 10 uM BIRB, followed by 60 minutes in DMSO, 25 uM 4HNE, 5 uM RSL3, 4HNE and BIRB, or RSL3 and BIRB. Isolated protein was assessed by immunoblotting for prominin2, phospho-HSF1 (S326), total HSF1, phospho-p38 (Thr180/Tyr182), total p38, and beta-actin. Shown is one replicate of three independent experiments. E) MCF10A cells were plated on slides pre-coated with laminin. Cells were then pretreated for 15 minutes with DMSO or 10 uM BIRB, followed by 60 minutes in DMSO, 25 uM 4HNE, 5 uM RSL3, 4HNE and BIRB, or RSL3 and BIRB. Cells were stained for total HSF1 and counterstained with DAPI. Images were taken at 20× magnification. Shown is one replicate of three independent experiments F) HSF1 expression was diminished in MCF10A cells for 48 hours using siRNA prior to treatment with DMSO, 25 uM 4HNE, or 5 uM RSL3. Isolated protein was assessed by immunoblotting for prominin2, phospho-HSF1 (Ser326), total HSF1, phospho-p38 (Thr180/Tyr182), total p38, and beta-actin. Shown is one replicate of three independent experiments. G) HSF1 expression was diminished in MCF10A cells for 48 hours using siRNA prior to treatment with DMSO or RSL3 at the range of concentrations shown. Cells were assessed for viability after 24 hours. Absorbance was normalized to DMSO control. Shown are three independent replicates with standard deviation.

FIGS. 16A-F. HSF1 inhibition prevents prominin2-mediated evasion of ferroptosis. A) MCF10A cells were treated for 60 minutes with DMSO, 25 uM 4HNE, 5 uM RSL3, 4HNE and 10 uM KRIBB11, or RSL3 and KRIBB11. mRNA was isolated and PROM2 expression was quantified by qPCR. Shown are three independent experiments with standard deviation. B) MCF10A cells were treated for 60 minutes with DMSO, 25 uM 4HNE, 5 uM RSL3, 4HNE and 10 uM KRIBB11, or RSL3 and KRIBB11. mRNA was isolated and HSF1 expression was quantified by qPCR. Shown are three independent experiments with standard deviation. C) MCF10A cells were plated on slides pre-coated with laminin. Cells were treated for 60 minutes with DMSO, 25 uM 4HNE, 5 uM RSL3, 4HNE and 10 uM KRIBB11, or RSL3 and KRIBB11. Cells were stained for total HSF1 and counterstained with DAPI. Images were taken at 20× magnification. Shown is one replicate of three independent experiments D) MCF10A cells were treated for 60 minutes with DMSO, 25 uM 4HNE, 5 uM RSL3, 4HNE and 10 uM KRIBB11, or RSL3 and KRIBB11. Isolated protein was assessed by immunoblotting for prominin2, phospho-HSF1 (Ser326), total HSF1, phospho-p38 (Thr180/Tyr182), total p38, and beta-actin. Shown is one replicate of three independent experiments. E) MCF10A cells were treated with RSL3, FIN56, IKE or erastin at the range of concentrations shown with either DMSO or 10 uM KRIBB11. Cells were assessed for viability after 24 hours. Absorbance was normalized to DMSO control. Shown are three independent replicates with standard deviation. F) MCF10A cells were treated with RSL3 at the range of concentrations shown with DMSO, 10 uM BIRB, 10 uM KRIBB11, 100 nM Dasatinib, or 25 uM Erlotinib. Cells were assessed for viability after 24 hours. Absorbance was normalized to DMSO control. Shown are three independent replicates with standard deviation.

FIGS. 17A-C. HSF1 inhibition sensitizes cancer cells to ferroptotic cell death. A) Hs578t cells were treated with RSL3 or IKE at the range of concentrations shown with either DMSO or 10 uM KRIBB11. Cells were assessed for viability after 24 hours. Absorbance was normalized to DMSO control. Shown are three independent replicates with standard deviation. B) SW620 and SW480 cells were treated with RSL3 at the range of concentrations shown with either DMSO or 10 uM KRIBB11. Cells were assessed for viability after 24 hours. Absorbance was normalized to DMSO control. Shown are three independent replicates with standard deviation. C) HCC1806, SF295 (glioblastoma), and NCI HH1975 (non-small cell lung cancer, NSCLC), cells were treated with RSL3 at the range of concentrations shown with either DMSO or 10 uM KRIBB11. Cells were assessed for viability after 24 hours. Absorbance was normalized to DMSO control. Shown are three independent replicates with standard deviation.

### DETAILED DESCRIPTION

Described herein is a cell biological mechanism of ferroptosis resistance that has broad implications for understanding and altering how cells respond, or fail to respond, to pro-ferroptotic conditions. A critical component of this mechanism is the pentaspanin protein prominin2, whose expression can be increased by pharmacological and physiological triggers of ferroptosis. The induction of prominin2 expression in response to ferroptotic stress promotes the formation of MVBs and exosomes that function to transport iron out of the cell and, consequently, impede ferroptosis. The existence of this mechanism suggests that cells have evolved a regulated means to evade ferroptosis that involves limiting the intracellular concentration of this potentially toxic metal in times of stress. Cells that cannot induce prominin2 and export ferritin are therefore highly sensitive to the induction of ferroptosis.

Free iron is essential for the execution of ferroptosis (Dixon et al., 2012; Dixon et al., 2014). The concept that intracellular iron levels and ferroptosis sensitivity can be modulated on short (<2 h) timescales through the export of ferritin from the cell via an MVB/exosome pathway had not been considered previously. Reducing ferroptosis sensitivity may be a physiological role for the process of MVB-mediated ferritin export observed previously (Truman-Rosentsvit M, 2018). The present results imply that cytosolic iron increases in response to GPX4 inhibition or ECM detachment, but that this iron can be captured, incorporated into MVBs, and exported from the cell if prominin2 expression is sufficient.

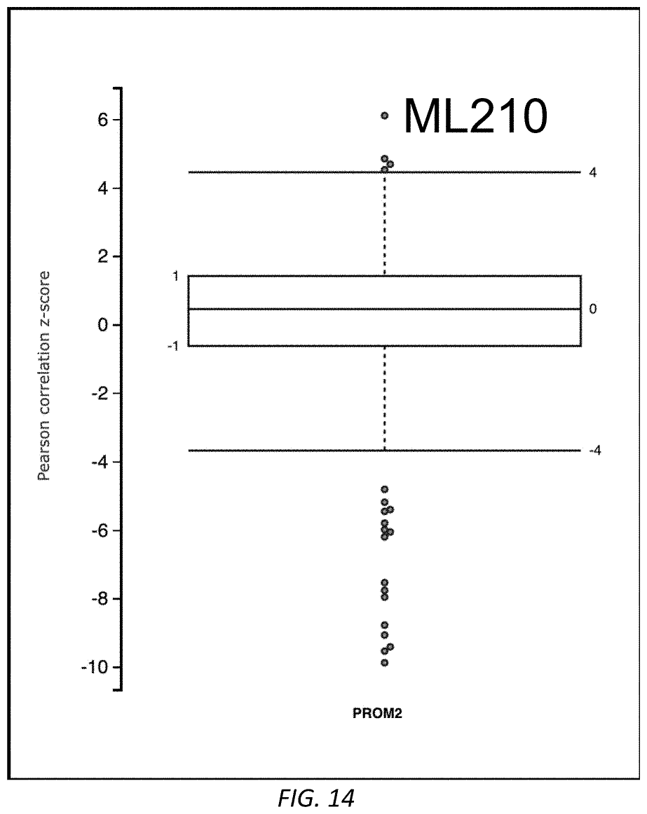

Until now, relatively little was known about the function or regulation of prominin2 in cells. The prominin2-mediated ferroptosis resistance described herein has implications for methods to induce ferroptosis as a mode of anti-cancer therapy. For example, inhibiting GPX4 activity can selectively kill certain tumor cells via ferroptosis (Viswanathan V S, 2017; Yang et al., 2014). Given that cells can acquire resistance to GPX4 inhibition by inducing prominin2, strategies that simultaneousy block prominin2 expression or function may enhance sensitivity to GPX4 inhibitors. Indeed, analysis of the Broad Institute CTRP resource indicated that, across hundreds of cancer cells lines, high levels of prominin2 expression are significantly correlated with resistance to the GPX4 inhibitor ML210 (portals.broadinstitute.org/ctrp/?featureName=PROM2). Prominin2 expression is correlated with poor clinical outcomes in several cancers (Saha et al., 2019); see FIG. 14. Prominin2-mediated MVB formation and iron export may also be relevant to the emerging role of ferroptosis in tumor suppression (Jiang et al., 2015).

Because prominin2 expression is essential to resisting ferroptoptotic cell death, we next sought to identify the mechanism by which prominin2 expression is regulated. We found that when a cell is experiencing ferroptotic stress via chemical inhibition of GPX4 etc., the transcription factor HSF1 is activated to induce prominin2. Inhibition of HSF1 by siRNA or chemically through KRIBB11 sensitized otherwise resistant cells to death induced by the compounds RSL3, FIN56, erastin, or imidazole ketone erastin.

Described herein are methods for overcoming tumor resistance to ferroptosis, and for triggering ferroptosis in tumor cells. The methods include administering one or more inhibitors of prominin2, optionally in combination with one or more promoters of ferroptosis, e.g., GPX4 inhibitors, to a subject in need thereof. The methods can also include administering one or more inhibitors of HSF1 in combination with one or more promoters of ferroptosis, e.g., GPX4 inhibitors, to a subject in need thereof.

Methods of Treatment

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers, in a subject. The subject can be a mammal, e.g., a human or non-human mammal such as a veterinary subject (e.g., a cat, dog, horse, goat, pig, or other animal).

In some embodiments, the disorder is a solid tumor, e.g., breast, prostate, pancreatic, brain, hepatic, lung, kidney, skin, or colon cancer. Generally, the methods include administering a therapeutically effective amount of a treatment as described herein, to a subject who is in need of, or who has been determined to be in need of, such treatment. In some embodiments, the subject is a candidate for use of a ferroptosis-inducing therapeutic, e.g., in cancers that depend on high levels of GSH or GPX4 for survival (such as the chemoresistant or “persister” cells described in Hangauer et al., 2017). In some embodiments, the methods include administering a therapeutically effective amount of a treatment comprising an inhibitor of PROM2, e.g., an inhibitory nucleic acid targeting PROM2, optionally in combination with one or more promoters of ferroptosis, e.g., GPX4 inhibitors, or a combination of an inhibitor of HSF1 in combination with one or more promoters of ferroptosis, e.g., GPX4 inhibitors. The treatment can be administered in combination with a standard treatment comprising chemotherapy, radiotherapy (e.g., as described in Lang et al., Cancer Discov 2019; 9:1673-85), and/or resection, and/or one or more promoters of ferroptosis, e.g., an inhibitor of glutathione peroxidase 4 (GPX4), or another treatment that promotes ferroptosis. In some embodiments the chemotherapy comprises one or more alkylating agents, antimetabolites, anti-microtubule agents, topoisomerase inhibitors, cytotoxic antibiotics, and/or platinum-based coordination complexes, e.g., cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib, sorafenib, sulfasalazine, ART, doxorubicin/Adriamycin (Lu et al., Front. Pharmacol., 12 Jan. 2018, doi.org/10.3389/fphar.2017.00992); US 20200138829.

As used in this context, to “treat” means to ameliorate at least one symptom of the disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, kidney, colon and ovary, including Clear-cell carcinomas (CCCs). The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CIVIL) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Inhibitors of Prom2

The present methods can include administration of one or more agents that inhibit Prom2. Such inhibitors can include inhibitory nucleic acids that target a PROM2 transcript. Alternatively or in addition, a PROteolysis TArgeting Chimera (PROTAC), comprising a Prom2 ligand linked to an E3 ubiquitin ligase (E3) recruiting ligand via a linker, can be used to degrade the Prom2 protein; see, e.g., Paiva and Crews, Curr Opin Chem Biol. 2019 June; 50:111-119; Sun et al., Sig Transduct Target Ther 4, 64 (2019). doi.org/10.1038/s41392-019-0101-6.

Inhibitors of HSF1

The present methods can include administration of one or more agents that inhibit HSF1. Such inhibitors can include inhibitory nucleic acids that target a HSF1 transcript, as well as KRIBB11 n(N(2)-(1H-indazole-5-yl)-N(6)-methyl-3-nitropyridine-2,6-diamine); Quercetin; Stresgenin B; Triplotide; Cantharidin; Fisetin; Rocaglamide A; 2,4-Bis(4-hydroxy benzyl)Phenol; CL-43; celasterol; cantharidin; 2,4-Bis(4-hydroxybenzyl)phenol; minnelide; KNK437; NZ-28; PW3405; IHSF115; 4,6-Disubstituted Pyrimidine; CCT361814; α-Acyl amino carboxamides; emunin; or bisamide (CCT251236) (see Sharma C, 2018). In some embodiments, the inhibitor is IHSF115 or KRIBB11.

Promoters of Ferroptosis/GPX4 Inhibitors

The present methods can include administration of one or more agents that promote ferroptosis. A number of such agents are known, including RSL3, Erastin, Imidazole ketone erastin (IKE), ML210, FIN56, Auranofin, Sulfasalazine, Artesunate, Artemisinin, Dihydroartemisinin, Sorafenib, Buthionine sulfoximine (BSO), Altretamine, Almitrine, and ML162, or an inhibitory nucleic acid molecule that targets GPX4. For example, the methods can include administering effective amounts of ascorbic acid (US20190216771); ultrasmall nanoparticles (US 20180169264); erastin or carbonyl erastin analogs (US 20160332974); quinazolinone inhibitors (US 20150175558); and the compounds described in US 20200138829, US 20190263802, and US 20190315681.

Inhibitory Nucleic Acids

Inhibitory nucleic acids useful in the present methods and compositions include antisense oligonucleotides, ribozymes, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target PROM2, HSF1, or GPX4 nucleic acid and modulate its function (e.g., reduce levels of transcript or transcription). In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target PROM2, HSF1, or GPX4 RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required. Exemplary target sequences for human PROM2, HSF1, and GPX4 are as follows.

PROM2 - Exemplary Human Sequences

| Nucleic acid | protein | Name |
|---|---|---|
| NM_144707.4 | NP_653308.2 | prominin-2 isoform 1 precursor |
| NM_001165977.3 | NP_001159449.1 | prominin-2 isoform 1 precursor |
| NM_001165978.3 | NP_001159450.1 | prominin-2 isoform 1 precursor |
| NM_001321070.2 | NP_001307999.1 | prominin-2 isoform 2 |

GPX4 - Exemplary Human Sequences

| Nucleic acid | protein | Name |
|---|---|---|
| NM_002085.5 | NP_002076.2 | phospholipid hydroperoxide |
| | | glutathione peroxidase isoform A |
| | | precursor* |
| NM_001039847.3 | NP_001034936.1 | phospholipid hydroperoxide |
| | | glutathione peroxidase isoform B |
| | | precursor |
| NM_001039848.4 | NP_001034937.1 | phospholipid hydroperoxide |
| | | glutathione peroxidase isoform C |
| NM_001367832.1 | NP_001354761.1 | phospholipid hydroperoxide |
| | | glutathione peroxidase isoform D |

*variant (1) represents the predominant transcript and encodes the canonical isoform A

HSF1 - Exemplary Human Sequences

| Nucleic acid | protein | Name |
|---|---|---|
| NM_005526.4 | NP_005517.1 | heat shock factor protein 1 |

Where the subject to be treating is a non-human mammal, a sequence from the species of that mammal can be used. Such sequences are readily identifiable using known methods.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within a PROM2, HSF1, or GPX4 RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA/shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc Natl Acad Sci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261:1411-1418; Szostak, 1993, MS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Ørom et al., Gene. 2006 May 10; 3720:137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)˜O˜CH2 (known as a methylene(methylimino) or MMI backbone], CH2-O—N(CH3)-CH2, CH2-N(CH3)-N(CH3)-CH2 and O—N(CH3)-CH2-CH2 backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH₃O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH₃; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, Helv. Chim. Acta, 1995, 78, 486). Other preferred modifications include 2′-methoxy (2′-0-CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-′7′7; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications', pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxgygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herien.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

Pharmaceutical Compositions

The methods described herein can include the administration of pharmaceutical compositions and formulations comprising inhibitory nucleic acid sequences designed to target a PROM2 RNA, an HSF1 RNA, and optionally one or more promoters of ferroptosis/inhibitors of GPX4.

In some embodiments, the compositions are formulated with a pharmaceutically acceptable carrier. The pharmaceutical compositions and formulations can be administered parenterally, topically, orally or by local administration, such as by aerosol or transdermally; preferably the administration is directly to a tumor. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. Preferably, the compositions are administered into or near a tumor, but they can also be administered systemically. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674. In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Krutzfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

Methods

The following materials and methods were used in the Examples set forth herein.

Experimental Model and Subject Details

Cell Lines: MCF10A cells were obtained from the Barbara Ann Karmanos Cancer Institute. Hs578T cells were provided by Dr. Dohoon Kim (UMASS Medical School). MDA-MB-231 cells were obtained from the American Type Culture Collection. All cells were checked quarterly for mycoplasma and authenticated using the University of Arizona Genetics Core.

The following antibodies were used: Prominin2, p-p38, total p38, beta-actin, 4HNE, pHSF1, total HSF1. Other reagents used were: 4HNE (Cayman Chemical), RSL3, erastin, FIN56 (SelleckChem), IKE (Med Chem Express), MDA (Sigma), KRIBB11 (Tocris. Med Chem Express), ferric ammonium citrate, ferrostatin-1 (Sigma).

RNA interference: siRNA-mediated silencing of gene expression of HSF1 was performed with Lipofectamine 3000 per the manufacturer's guidelines using pooled control or HSF1 siRNAs (Santa Cruz Biotechnology). Cells were transfected and after 48 hours were plated for experimental use.

Cell-based assays: Assays to assess survival in response to matrix detachment were performed as described (Brown C W, 2017). To assay survival in response to GPX4 inhibition, adherent cells in 96 well plates (1.0×10⁴ per well) were treated with either DMSO, RSL3, ML210, FIN56 or erastin for 24 hrs with and without ferrostatin-1 or DFO. Subsequently, cells were fixed in 4% paraformaldehyde (Boston BioProducts), stained with crystal violet (Sigma) and read on a microplate reader (Beckman Colter) at absorbance 595. Absorbances were normalized to DMSO.

Biochemical experiments: Immunoblotting and qPCR were performed as described (Brown C W, 2017). Briefly, for immunoblotting, cell lysates were extracted using radioimmunoprecipitation assay (RIPA) buffer (Boston Bioproducts). Extracts were separated by SDS PAGE using the specified antibodies. Immunoprecipitation was performed on lysates isolated using NP-40 buffer (Boston Bioproducts). Lysates were pre-cleared prior to overnight incubation with the primary antibody and protein A agarose beads (Sigma-Aldrich). Immune complexes were separated by SDS PAGE and immunoblotted as described in figure legends. RNA was isolated from cells (BioBasic) and cDNA was generated using the All in One cDNA Synthesis SuperMix (BioScript Solutions). qPCR was performed using a SYBR green master mix (BioScript Solutions). Sequences for primers used were as follows:

18s Forward
(SEQ ID NO: 1)
5′-AACCCGTTGAACCCCATT-3′,
18s Reverse
(SEQ ID NO: 2)
5′-CCATCCAATCGGTAGTAGCG-3′,
PROM2 Forward
(SEQ ID NO: 3)
5′-GCTCAGGAACCCAAACCTGT-3′,
PROM2 Reverse
(SEQ ID NO: 4)
5′-GGCAGGCCATACATCCTTCT-3′,
PROM1 Forward
(SEQ ID NO: 5)
5′-AGTCGGAAACTGGCAGATAGC-3′,
PROM1 Reverse
(SEQ ID NO: 6)
5′-GGTAGTGTTGTACTGGGCCAAT-3′.

Total free iron was quantified using the Iron Assay Kit (Abcam) per manufacturer's specifications. Briefly, adherent cells in 10 cm plates (1.0×10⁶/well) were treated with either DMSO or RSL3 for 2 hrs, lysed and supernatant was reduced using the provided iron reducer before the mixture was reacted with an iron probe. Plates were read at 595 nm and total iron concentration was obtained using an iron standard curve.

Molecular biology experiments siRNA-mediated silencing of gene expression was performed with Lipofectamine 3000 (Invitrogen) per the manufacturer's guidelines using control, prominin2, TSG101, or FTH1/FTL siRNAs (Santa Cruz Biotechnology; sc-94521, sc-36752, sc-40575/sc-40577 respectively). To express prominin2 in MDA-MB-231 cells, a pCMV-prominin2 expression vector was obtained from VectorBuilder. Cells were transfected with this plasmid and a control plasmid using Lipofectamine 3000 (ThermoFisher) and selected for prominin2 expression with G418 (0.5 μg/mL) (Gibco) for ten days.

Immunofluorescence microscopy: For adherent cells, 8 well chamber slides (Mattek) were coated with 1 μg/mL laminin (Invitrogen). Cells (5.0×10⁴ per well) were plated overnight and then treated for two hours with either DMSO or RSL3, fixed in 4% paraformaldehyde and permeabilized in 0.1% Triton (JT Baker). Slides were blocked in 0.5% BSA (Boston Bioproducts), incubated in antibody overnight at 4° C., washed in PBS and incubated in secondary antibody for 1 hr at room temperature. Slides were washed and mounted in Vectashield with DAPI (Vector Labs). For ECM-detached experiments, cells were trypsinized and plated (5.0×10⁵ per well) in 60 mm low-adherent plates (Sumitomo Bakelite) for two hr, collected and spun down prior to being fixed and stained as described above. Slides were imaged on a Zeiss or a Nikon A-1 confocal microscope.

Exosome isolation: Cells were cultured for 24 hrs in their respective media containing extracellular vesicle (EV) free serum and treated with RSL3 for 24 hours. Culture supernatants were centrifuged at 4° C. at 1000 RPM for 5 min, 3000 RPM for 10 min, 4000 RPM for 30 min prior to ultracentrifugation at 22,000 RPM for 140 min at 4° C. Pellets were resuspended in cold PBS and centrifuged again under the same conditions. For immunoblotting, pellets were lysed in RIPA buffer, centrifuged at 10,000 RPM for 15 min and protein concentration was determined by Bradford Assay (BioRad). For TEM, PBS was removed and 20 μL of the exosome preparation was fixed and processed. For ICP-mass spec, exosome-containing pellet was resuspended in 0.5 mL PBS and centrifuged in a speed-vac at 45° C. Samples were sent to the UMASS Mass Spectrometry Core where they were digested in 100 μL HNO₃, heated to 65° C. for 20 min and diluted to 2 mL with ddH₂O before analysis.

Transmission Electron Microscopy: Adherent siControl and siProminin2 cells were treated with DMSO or RSL3 (5 μM) for two hr. ECM-detached siControl and siProminin2 cells were maintained for 2 hrs in low-adherence plates. All cells were fixed by adding 2.5% glutaraldehyde (v/v) in 0.1 M Na cacodylate buffer (pH 7.2) and processed for TEM. The samples were examined on a FEI Tecnai 12 BT transmission electron microscope using 100 Kv accelerating voltage. Images were captured using a Gatan TEM CCD camera.

Statistical analysis: All experiments were independently repeated at least three times. Statistical analysis was performed using GraphPad Prism software. The p-value was calculated using Student's t test or one way ANOVA and a p-value <0.05 was considered significant.

### Example 1. Prominin2 is Induced by Ferroptotic Stress and Promotes Resistance to Ferroptotic Cell Death

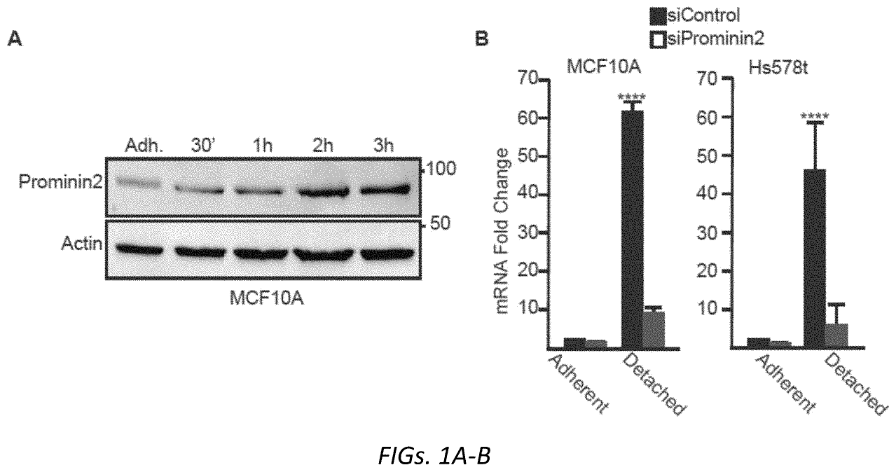

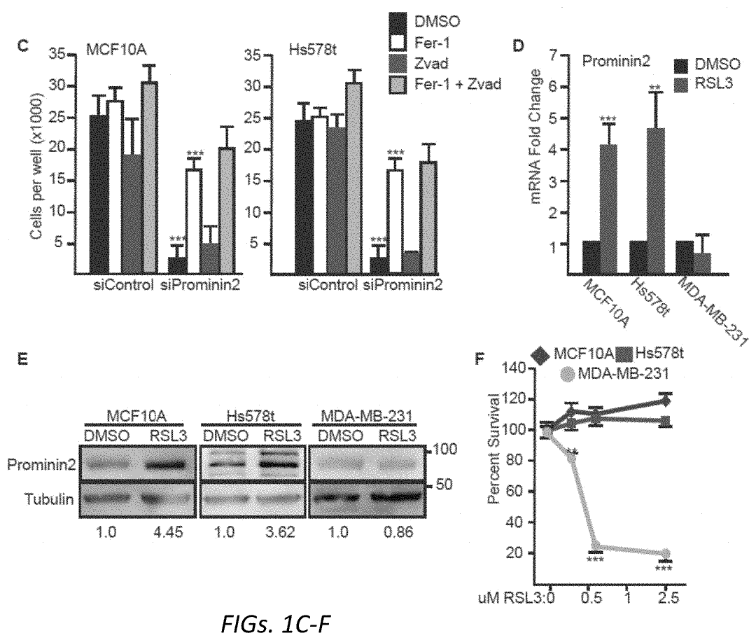

To gain insight into mechanisms that promote ferroptosis resistance, we exploited our recent finding that detachment of certain cells from the extracellular matrix (ECM) is a pro-ferroptotic stress (Brown, 2018; Brown C W, 2017). We reasoned that cells that can survive under detached conditions may do so by upregulating a protective gene expression program that prevents cell death. To identify such genes, we compared mRNA expression in adherent and ECM-detached MCF10A immortalized breast epithelial cells by RNA-sequencing (GSE115059). PROM2, which encodes the pentaspanin protein prominin2, captured our attention because its expression was significantly increased in ECM-detached cells compared to adherent cells. Also, it is thought to have a role in lipid dynamics and membrane organization, processes that could be relevant for ferroptosis resistance (Doll S, 2017; Florek M, 2007; Jászai J, 2010; Singh R D, 2013). We confirmed in MCF10A cells that prominin2 was rapidly upregulated in response to ECM detachment at the protein and mRNA level (FIGS. 1A,B). Increased PROM2 was also observed in detached versus attached Hs578t breast cancer cells (FIG. 1B). Silencing of prominin2 expression in both MCF10A and Hs578t cells resulted in a significant loss of viability in ECM-detached conditions (FIGS. 1B,C). Cell viability under these conditions was rescued by co-treatment with a specific inhibitor of ferroptosis, ferrostatin-1 (Dixon et al., 2012), but not the pan-caspase inhibitor ZVAD-fmk (FIG. 1C). These data indicate that failure to upregulate PROM2 upon ECM detachment can result in the induction of ferroptosis.

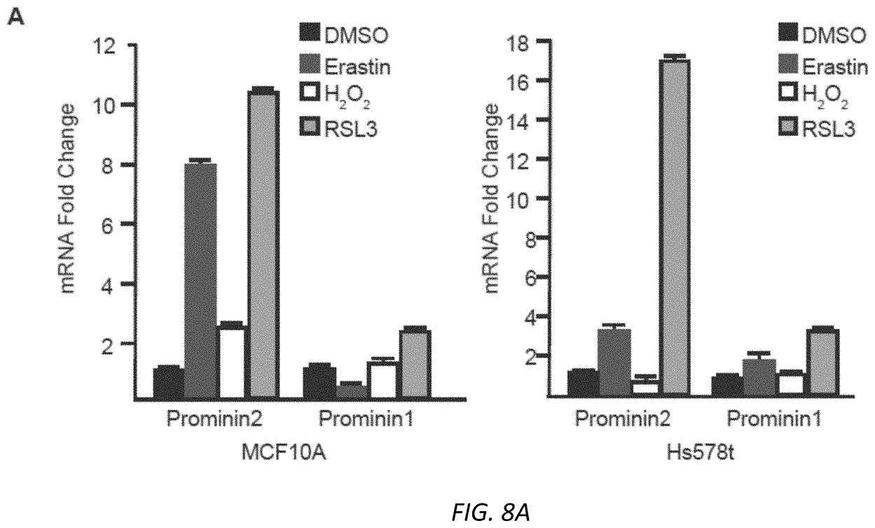

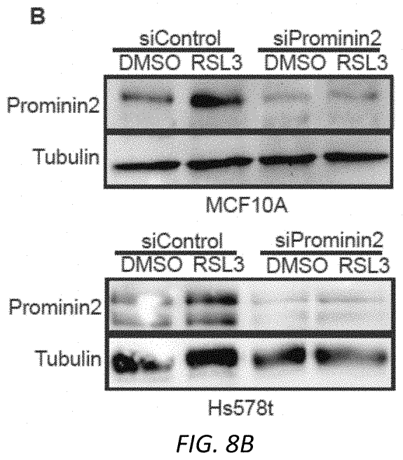

We next investigated whether pharmacological inhibition of GPX4 altered prominin2 expression and impacted ferroptosis in adherent cells. MCF10A and Hs578t cells were treated with RSL3, a covalent GPX4 inhibitor (Yang et al., 2014), for 2 hrs resulted in increased prominin2 mRNA and protein expression in both cell lines (FIGS. 1D,E). This effect was not a generic response to ROS stress because culturing with H₂O₂ did not increase prominin2 expression in either MCF10A or Hs578t cells (FIG. 8A). Moreover, expression of a related family member, prominin1 (CD133), was not altered significantly by RSL3, further demonstrating the specificity of the ferroptotic effect for prominin2 (FIG. 8A). Unlike MCF10A and Hs578t cells, RSL3 did not induce prominin2 in MDA-MB-231 cells, another breast cancer cell line (FIGS. 1D,E). We observed that the induction of prominin2 expression correlated with resistance to GPX4 inhibitor-induced ferroptosis: MCF10A and Hs578t cells were resistant to RSL3-induced cell death at concentrations up to 2.5 while MDA-MB-231 cells were killed by RSL3 concentrations as low as 0.5 μM (FIG. 1F).

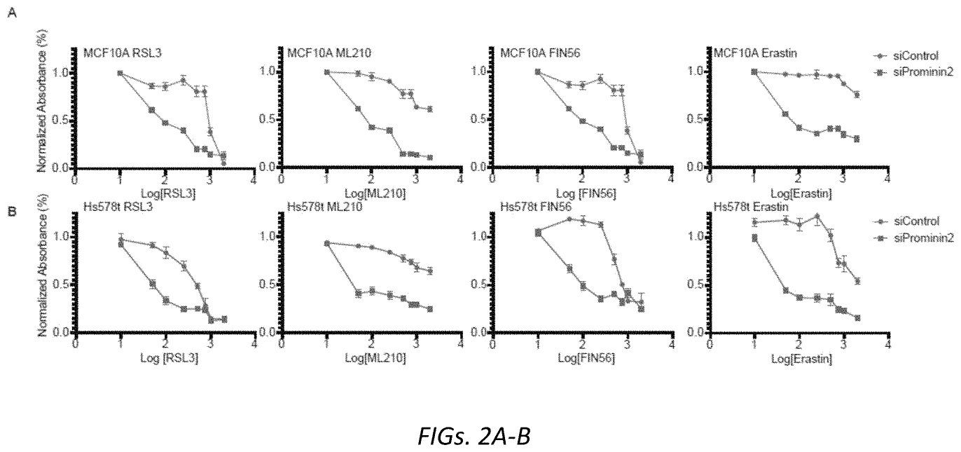

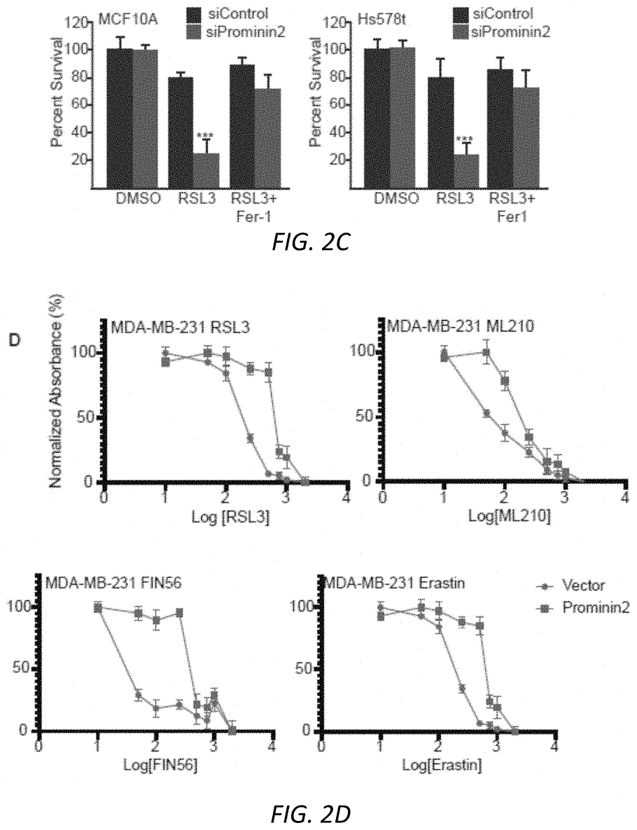

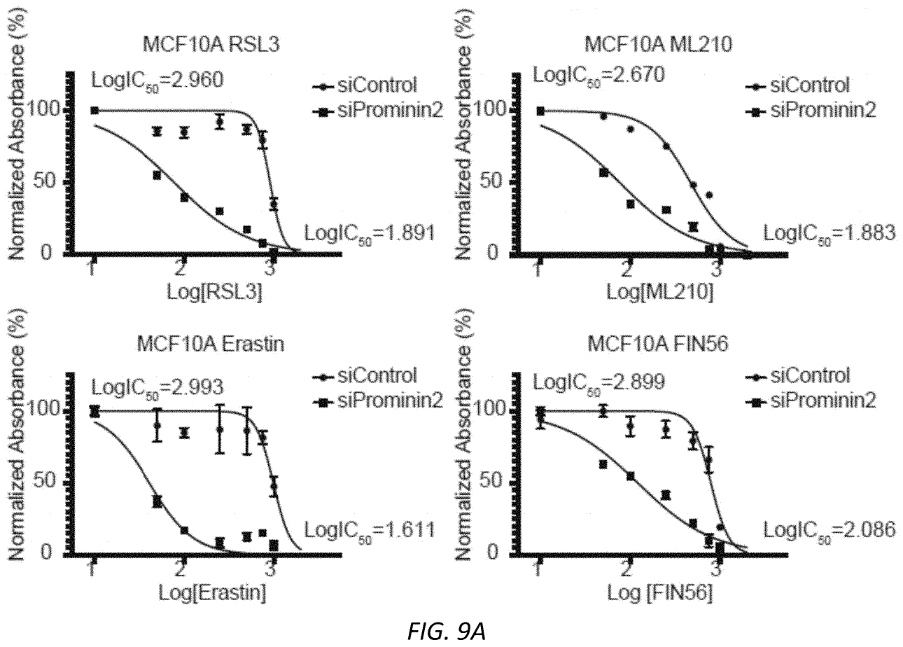

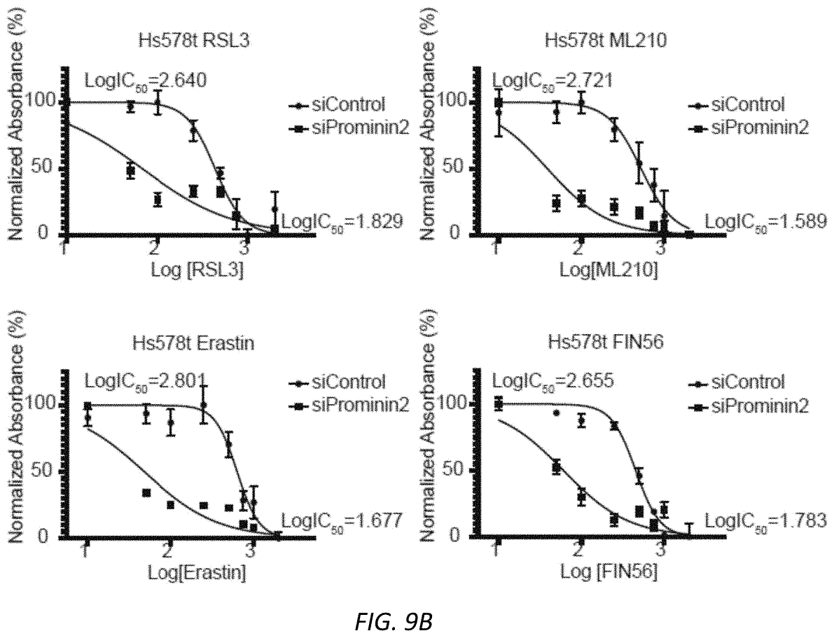

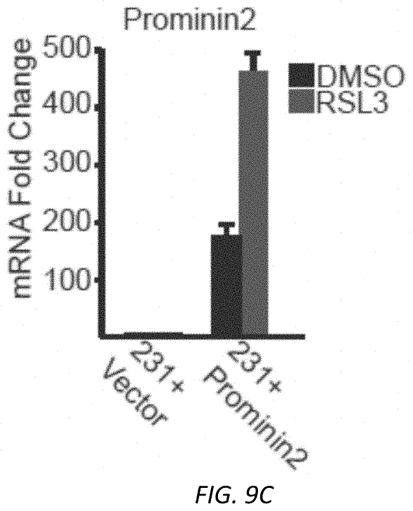

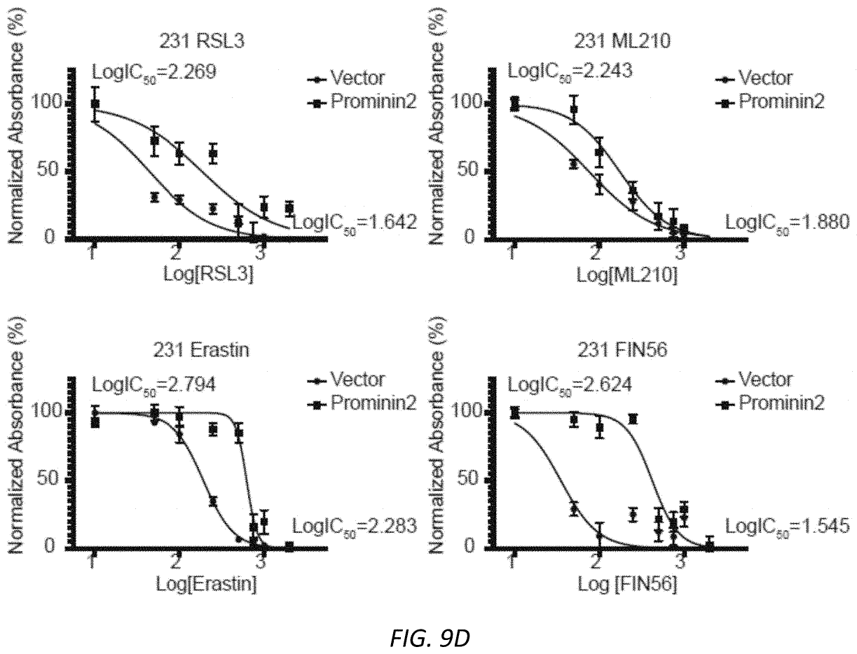

Given the above results, we assessed whether prominin2 was necessary for resistance to ferroptosis. For this purpose, we silenced prominin2 expression in MCF10A and Hs578t cells (FIG. 8B) and examined the sensitivity of these cells to ferroptosis compared to control cells. Here, we tested the covalent GPX4 inhibitors RSL3, ML210 (Weïwer M, 2012), the indirect GPX4 inhibitor FIN56 (Shimada K, 2016), and the GSH-depleting agent erastin, which reduces GPX4 activity indirectly by preventing the synthesis of its key cofactor (Dixon et al., 2012). Control MCF10A cells were more resistant to these compounds than Hs578t cells (FIGS. 2A,B). Nonetheless, a significant increase in sensitivity to all four compounds was observed in prominin2 knock-down cells compared to control cells for both cell lines (FIGS. 2A,B, 9A,B). Loss of cell viability in prominin2-depleted cells was completely rescued by ferrostatin-1, indicating that the greater sensitivity of prominin2-silenced cells was not caused by the induction of an alternative mode of cell death (FIG. 2C). Given the results obtained with genetic silencing, we next tested whether overexpression of prominin2 was sufficient to inhibit ferroptosis. Indeed, in MDA-MB-231 cells exogenous prominin2 expression was sufficient to inhibit cell death in response to RSL3, ML210, FIN56 and erastin (FIGS. 2D, 9C,D). Thus, prominin2 expression promotes resistance to ferroptosis. Subsequent experiments were performed using RSL3 as a prototypic GPX4 inhibitor.

### Example 2. Prominin2 Promotes the Formation of Multivesicular Bodies and Exosomes

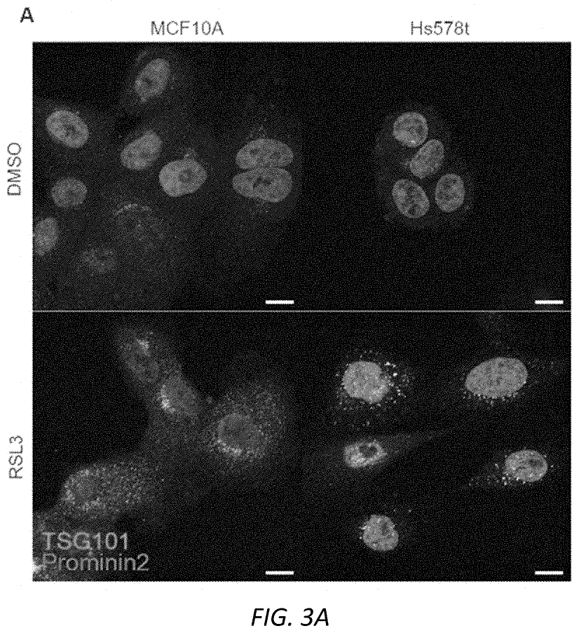

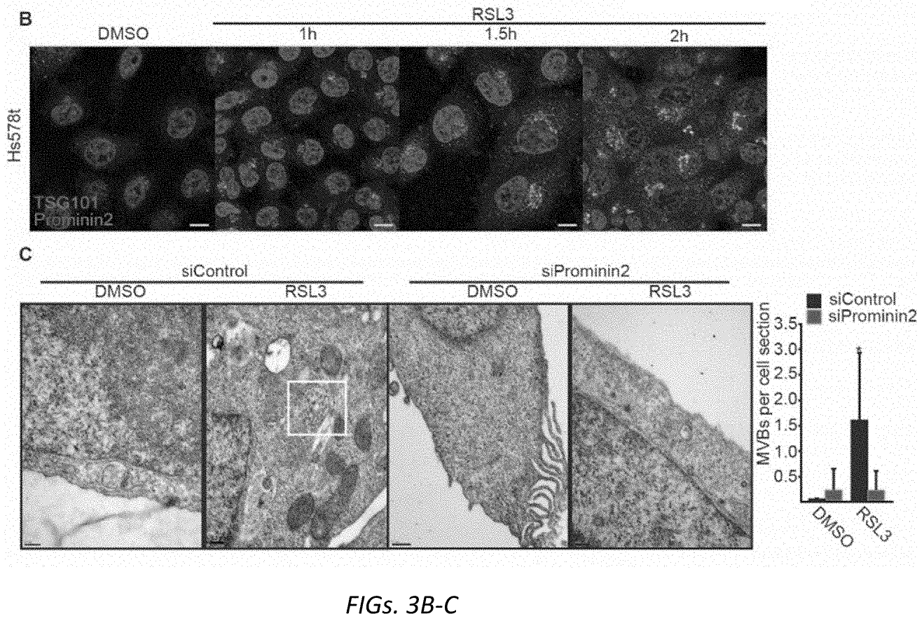

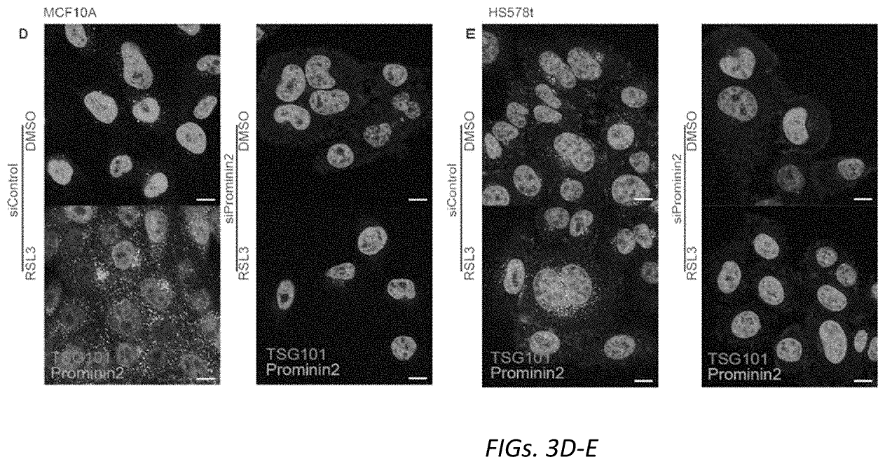

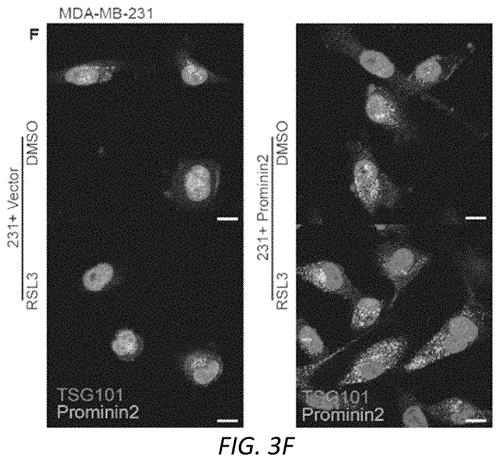

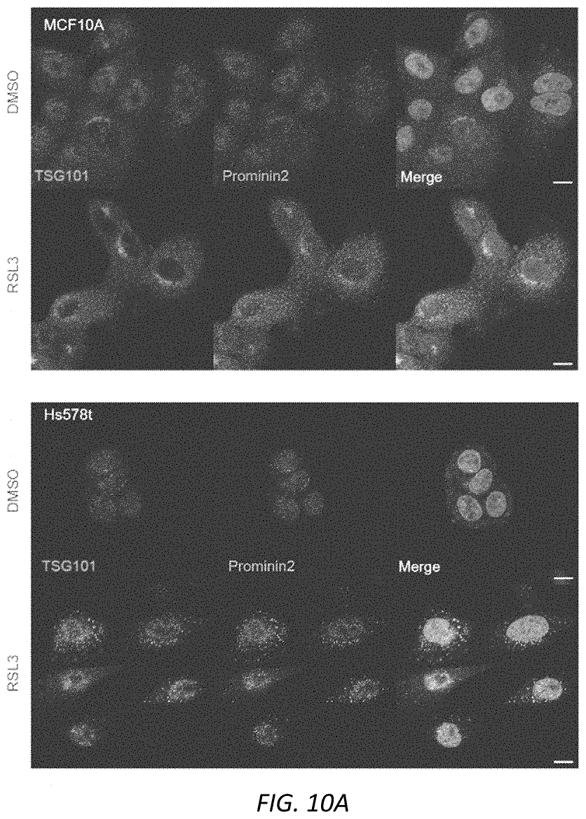

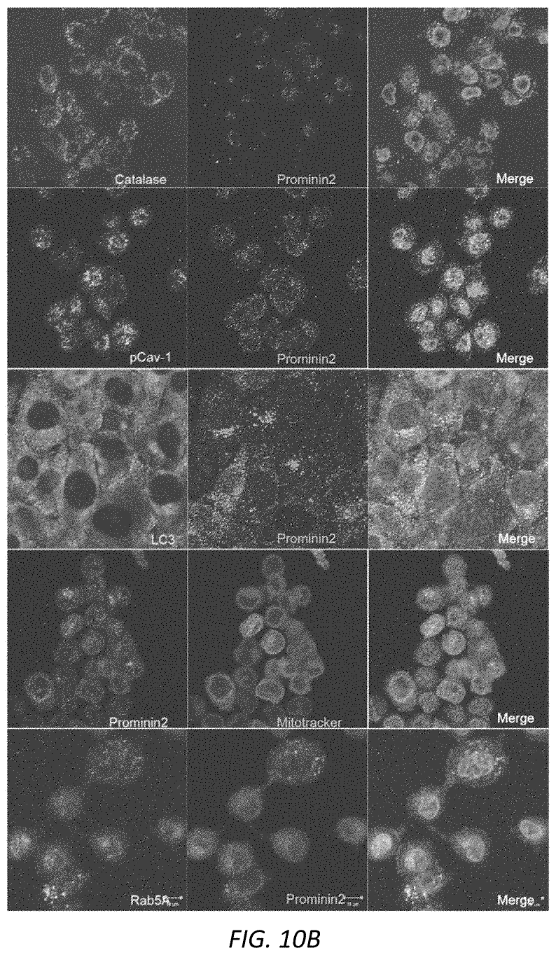

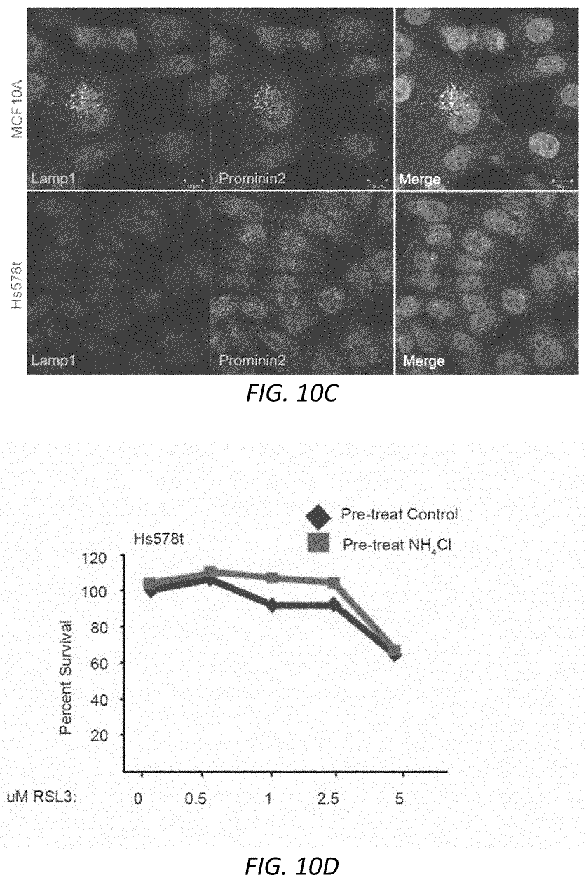

To gain insight into how prominin2 may promote ferroptosis resistance, we assessed its subcellular localization following GPX4 inhibition. Immunofluorescence microscopy revealed that prominin2 localizes in discrete cytoplasmic puncta in response to GPX4 inhibition by RSL3 (FIGS. 3A, 10A). As determined using organelle-specific markers, these puncta did not co-localize with early endosomes (Rab5A), peroxisomes (catalase), caveolae (phospho-Caveolin-1), autophagosomes (LC3) or mitochondria (Mitotracker) (FIG. 10B). By contrast, a striking co-localization was observed between prominin2 and TSG101, a marker of multivesicular bodies (MVBs) (Razi M, 2006) (FIGS. 3A, 10A). MVBs are late endosomes containing cargo within their lumen bound in intraluminal vesicles (ILVs) (Felder S, 1990; Gruenberg J, 1995). The cargo can be sorted for degradation in lysosomes or released into the extracellular space as exosomes (Futter C E, 1996; Hart P D, 1991). The co-localization of prominin2 and TSG101 was evident as early as 30 min following the inhibition of GPX4 and this co-localization was most prominent in the perinuclear region (FIG. 3B, 11A). At later times (1.5 to 2 hrs), this co-localization was evident throughout the cytoplasm. We also observed that prominin2 exhibited some co-localization with the lysosomal marker LAMP1 (Cella N, 1996) (FIG. 10C), which is expected given that some MVBs are trafficked to lysosomes (Futter C E, 1996; Razi M, 2006). However, we discounted a role for lysosomes in ferroptosis resistance under these conditions based on the observation that NH₄Cl treatment, which inhibits lysosome function (Hart P D, 1991), had no effect on cell survival following GPX4 inhibition (FIG. 10D).

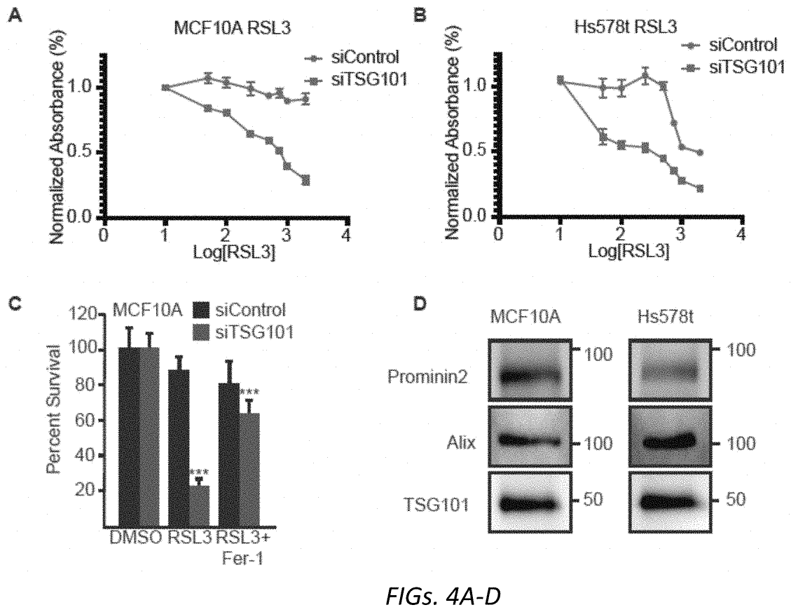

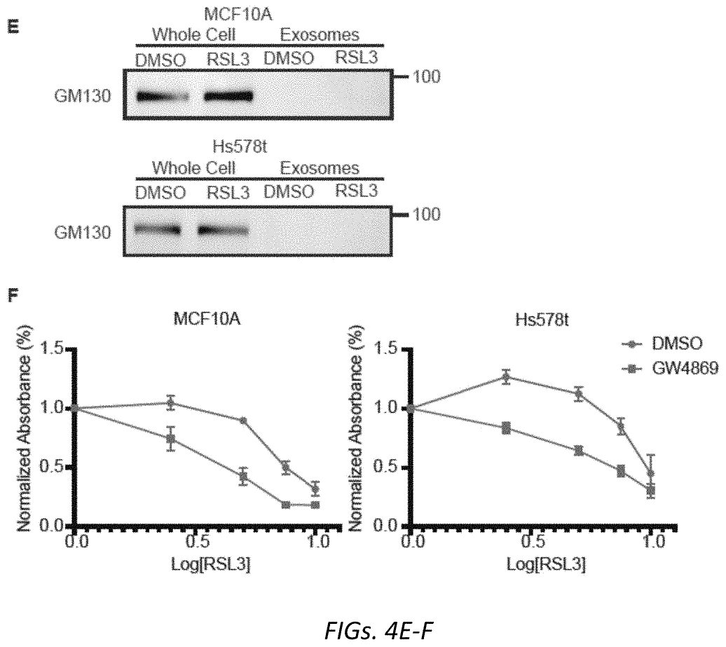

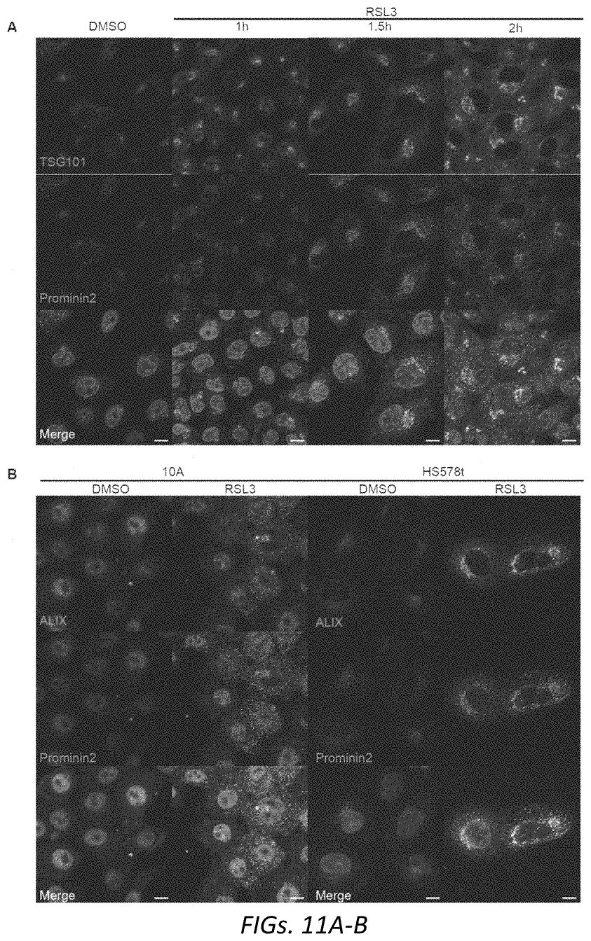

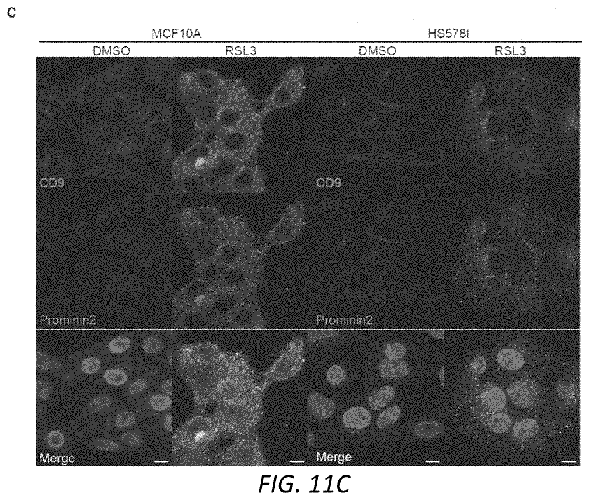

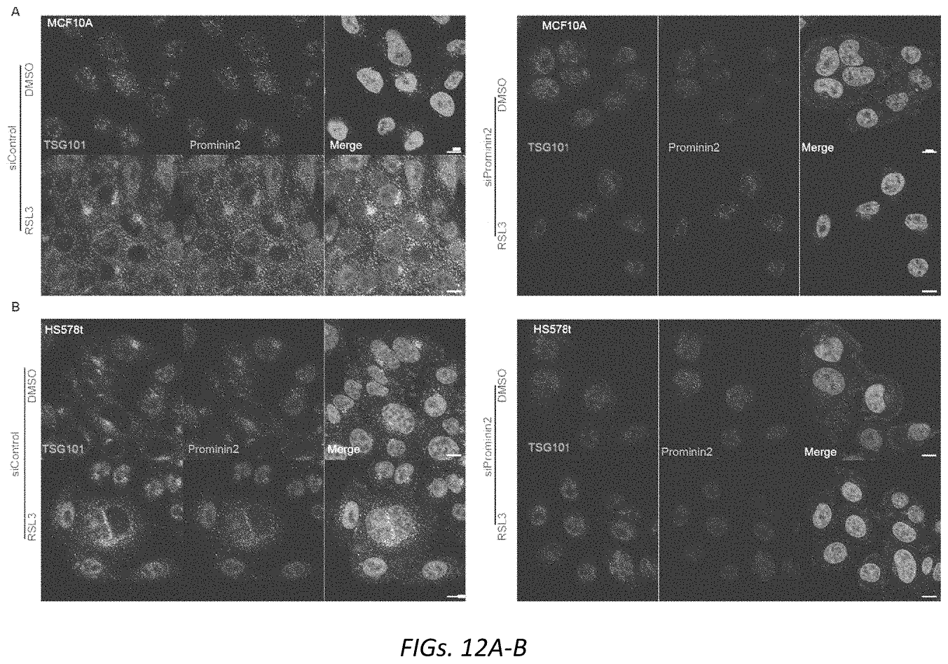

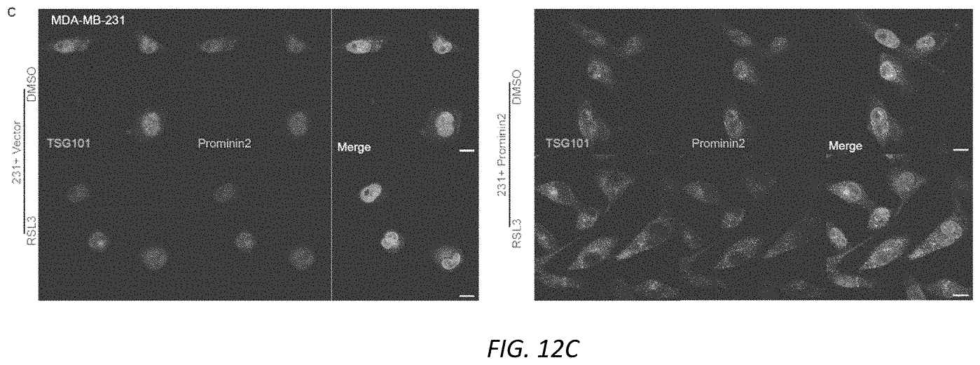

The above results prompted us to assess whether prominin-2 promotes MVB formation in response to GPX4 inhibition. RSL3-treated MCF10A and Hs578t cells contained more TSG101-positive structures than vehicle control-treated cells, indicating that GPX4 inhibition induces MVB formation (FIGS. 3A,B, 11A). Prominin2 localization to MVBs in RSL3-treated MCF10A and Hs578t cells was confirmed by co-localization with Alix and CD9 (FIGS. 11B, C). We then evaluated whether the formation of these structures was dependent on prominin2 using transmission electron microscopy (TEM). Indeed, RSL3-treated MCF10A cells contained significantly more MVBs per cell than control cells, an effect that was not observed in prominin2-silenced cells (FIG. 3C). Notably, this RSL3-stimulated increase in MVBs was rapid (within 2 hr). Immunofluorescence microscopy also revealed a reduction in TSG101-positive puncta in prominin2-depleted MCF10A and Hs578t cells compared to control cells (FIGS. 3D,E, 12A,B). Conversely, exogenous prominin2 expression in MDA-MB-231 cells increased the number of TSG101-positive structures and, as noted above, reduced ferroptosis sensitivity (FIGS. 3F, 12C). Consistent with a causal role for MVBs in ferroptosis resistance, diminishing TSG101 expression increased the sensitivity of MCF10A and Hs578t cells to RSL3-induced cell death (FIGS. 4A,B, 13A) and this death was rescued with ferrostatin-1 (FIG. 4C). These data indicate that GPX4 inhibition stimulates prominin2 expression and prominin2-dependent MVB formation, which promote ferroptosis resistance.

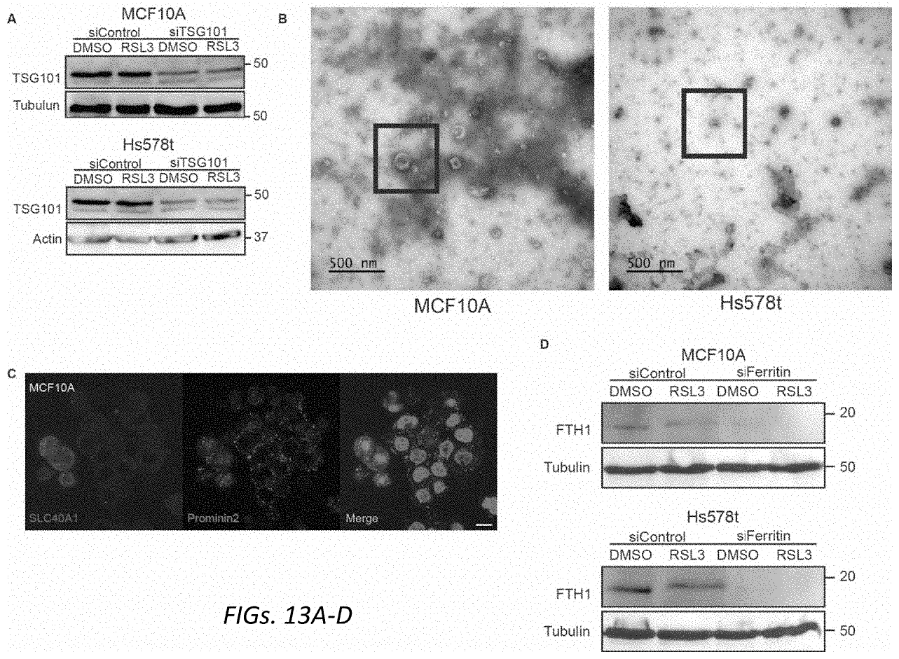

MVBs can fuse with the plasma membrane and release intraluminal vesicles (ILVs) as exosomes (Harding C, 1984; Pan B T, 1985). Indeed, we observed distinct ILVs in MVBs consistent with the size of exosomes (˜80 nm) (Raposo and Stoorvogel, 2013) in RSL3-treated cells (FIG. 3C). To assess the impact of GPX4 inhibition on exosome formation, exosomes were harvested by ultracentrifugation from control (DMSO) and RSL3-treated MCF10A and Hs578t cells. The presence of an exosome fraction was confirmed by TEM (FIG. 13B). GPX4 inhibition increased exosome formation as evidenced by the fact that a significant amount of protein was present in the exosome preparation of RSL3-treated cells (FIG. 4D). In contrast, exosomal protein in control cells was not detected in this experiment (data not shown). Analysis of these exosomes by immunoblotting revealed that they contained prominin2, Alix and TSG101 providing evidence that they were generated by prominin2-dependent MVB formation (FIG. 4D). Importantly, they did not contain GM130, a Golgi marker, indicating that their isolation was not contaminated with other cell membranes (FIG. 4E). To test whether exosome release was essential for resistance to GPX4 inhibition, we used the sphingomyelinase inhibitor GW4869 and found that inhibition of exosome release increased the sensitivity of MCF10A and Hs578t cells to RSL3 (FIG. 4F).

### Example 3. Ferroptosis Resistance is Facilitated by MVB/Exosome Iron Transport

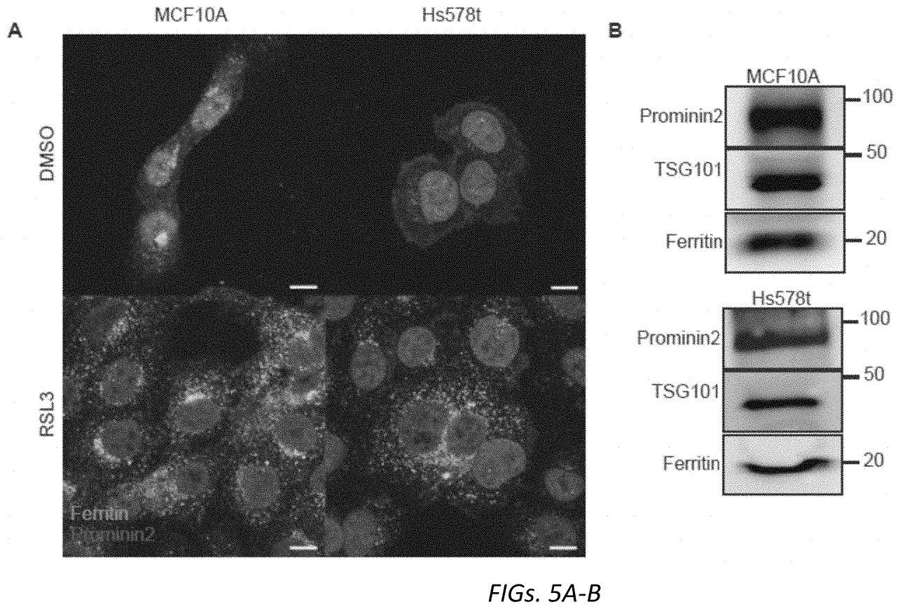

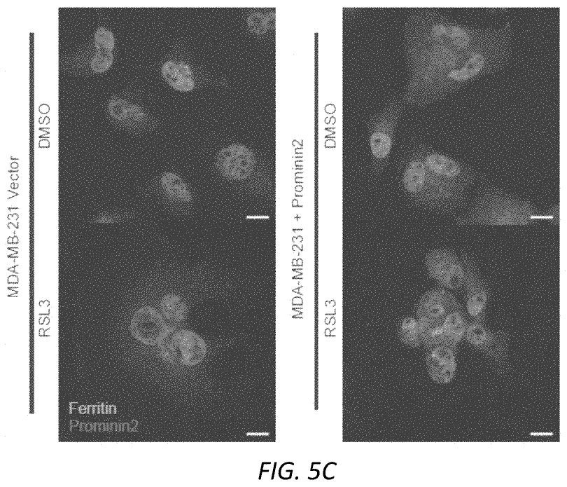

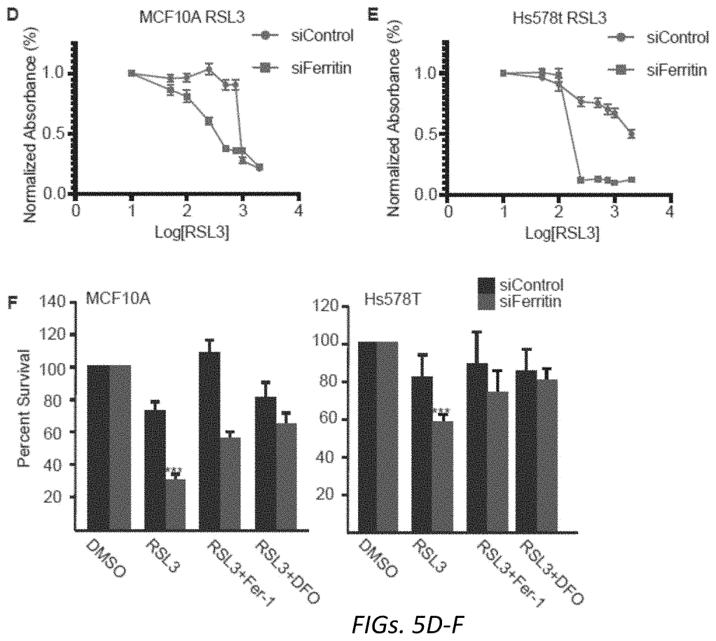

Our results indicated that prominin2-mediated MVB and exosome formation promote ferroptosis resistance. We hypothesized that this resistance mechanism involved MVBs and exosome-mediated export of a factor that would otherwise promote ferroptosis. Given that the iron storage protein ferritin can be secreted from cells in exosomes (Truman-Rosentsvit M, 2018), we hypothesized that MVB/exosome-mediated ferritin export inhibits ferroptosis. In support of this hypothesis, we observed that ferritin co-localized with prominin2 in RSL3-treated MCF10A and Hs578t cells, but not in vehicle control (DMSO)-treated cells (FIG. 5A). We also identified ferritin protein in exosomes harvested from these cells (FIG. 5B). These exosomes contained iron as detected by inductively coupled plasma mass spectrometry (ICP-MS) (3400 ng per exosome collection sample), further supporting our hypothesis. MDA-MB-231 cells do not increase ferritin expression following GPX4 inhibition. However, the expression of exogenous prominin2 induced a more punctate pattern of ferritin localization in RSL3-treated compared to control cells (FIG. 5C). An alternative explanation for our findings was that prominin2 enhanced the membrane localization of the iron exporter ferroportin, which has been shown to play a role in ferroptosis sensitivity (Geng N, 2018). However this did not appear to be the case because prominin2 did not co-localize with ferroportin (SLC40A1) (FIG. 13C).

A causal role for ferritin in ferroptosis resistance is supported by the finding that knock-down of both the ferritin heavy chain (FTH1) and light chain (FTL) increased cell death triggered by GPX4 inhibition in both MCF10A and Hs578t cells (FIGS. 5D,E, 13D). Importantly, this death was rescued by either ferrostatin-1 or the iron chelator deferoxamine (DFO), providing evidence for its ferroptotic nature (FIG. 5F).

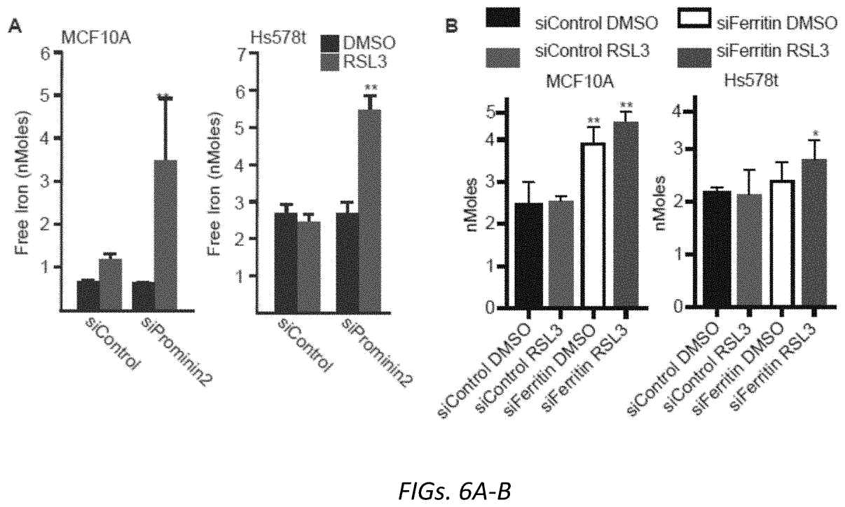

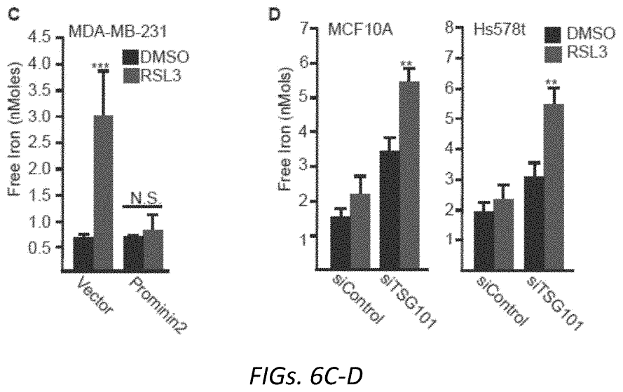

Our data suggested that GPX4 inhibition stimulates the formation of MVBs containing ferritin and that these structures transport iron out of the cell. To test this hypothesis, we quantified the free iron concentration in control and RSL3-treated MCF10A and Hs578t cells and observed no significant difference between these two groups (FIG. 6A). However, when either prominin2 or both FTH1 and FTL were silenced, we observed an increase the concentration of free iron following RSL3 treatment (FIGS. 6A,B, S6D). Additional evidence for prominin2 regulation of iron levels was obtained by quantifying free iron in control and prominin2-expressing MDA-MB-231 cells in response to GPX4 inhibition. Prominin2 expression in these cells prevented the increase in iron in response to RSL3 that was observed in control cells (FIG. 6C). Also, disabling the MVB pathway by decreasing expression of TSG101 significantly increased the concentration of free intracellular iron following GPX4 inhibition (FIG. 6D). These results indicate that blocking MVB/exosome-mediated ferritin export results in an accumulation of intracellular iron upon GPX4 inhibition.

### Example 4. ECM-Detached Cells Resist Ferroptosis by Prominin2-Mediated MVB/Exosome Formation

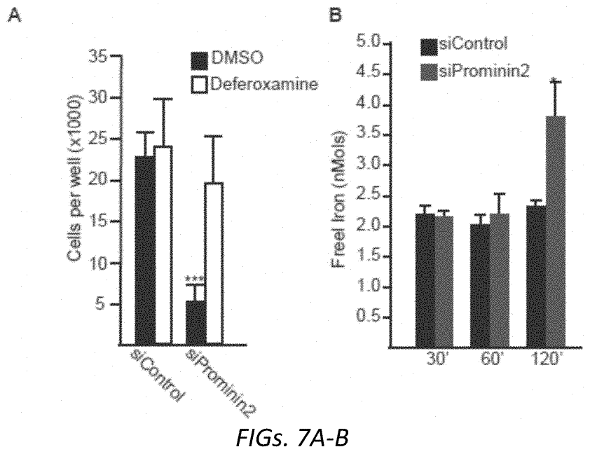

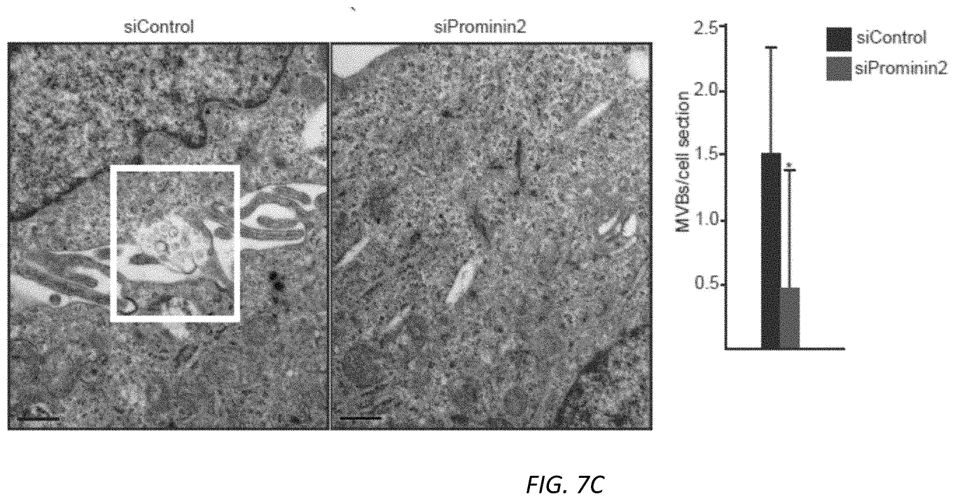

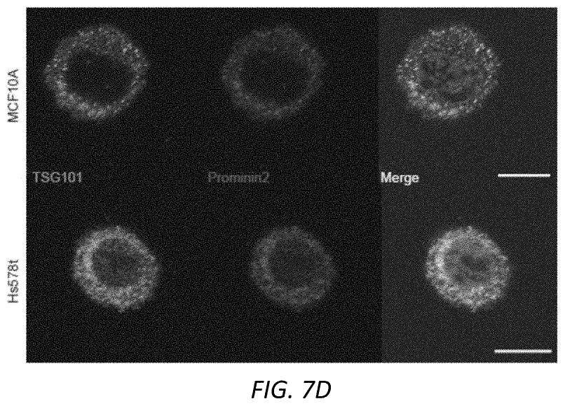

Based on the observation that ECM-detachment is a ferroptotic stress that induces prominin2 expression (FIG. 1B), we investigated whether prominin2 promotes MVB formation and iron transport in ECM-detached cells. Indeed, cell death in ECM-detached, prominin2-depleted MCF10A and Hs578t cells was rescued by the iron chelator deferoxamine, consistent with a defect in iron export promoting cell death (FIG. 7A). Also, ECM detachment increased the concentration of free iron in prominin2-depleted compared to control cells, an effect that was most noticeable 2 hrs post-detachment, which is coincident with the onset of ferroptosis (FIG. 7B). Similar to RSL3-treated adherent cells, ECM detachment increased the frequency of MVBs based on TEM analysis and this increase was dependent on prominin2 (FIG. 7C). Exosome release is indicated by the fusion of an MVB with the plasma membrane (FIG. 7C). Co-localization of prominin2 and TSG101 was also evident in ECM-detached MCF10A and Hs578t cells (FIG. 7D), similar to the co-localization seen in adherent, RSL3-treated cells (FIG. 3A). These results indicate that physiological ferroptotic stress can be evaded by the prominin2/MVB iron export pathway.

### Example 5. Ferroptosis Resistant Cancer Cells Succumb to HSF-1 Inhibition

As noted above, depletion of prominin2 expression renders resistant cells vulnerable to ferroptosis. To gain further insight into this process, we explored the mechanism by which prominin2 expression is induced by ferroptotic stress, to reveal targets for additional therapeutic intervention.

In this study, we discovered that a metabolite resulting from degraded, peroxidated lipids, 4-hydroxynonenal (4HNE) stimulates PROM2 transcription by a mechanism that is dependent upon heat shock factor 1 (HSF1). Furthermore, we demonstrate that currently available HSF1 inhibitors overcome resistance to GPX4 inhibition and significantly increase the sensitivity of cancer cells to ferroptosis. This finding increases our understanding of mechanisms used by cells to resist ferroptosis and it has potentially valuable implications for therapies aimed at stimulating ferroptosis.

### Example 5.1. Induction of PROM2 mRNA by 4HNE is Mediated by HSF-1

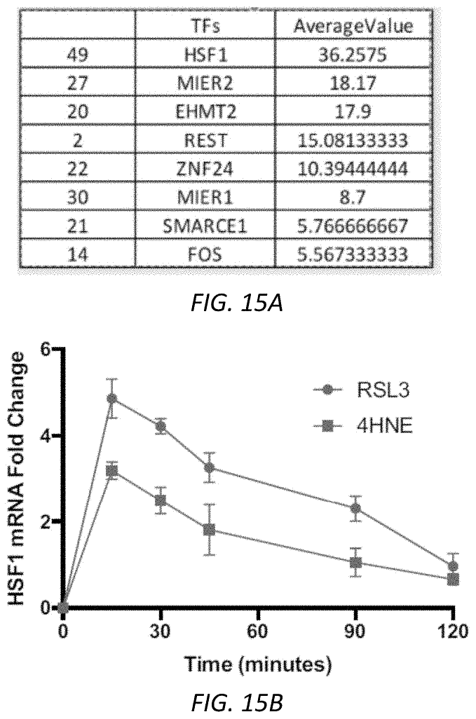

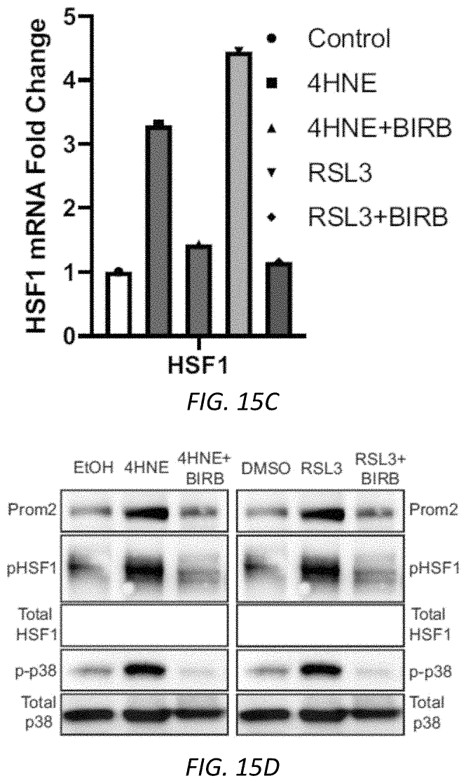

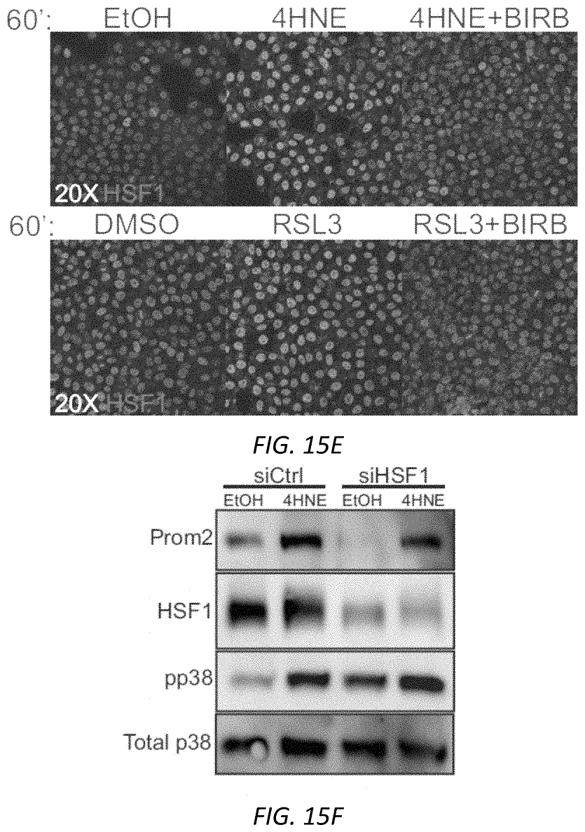

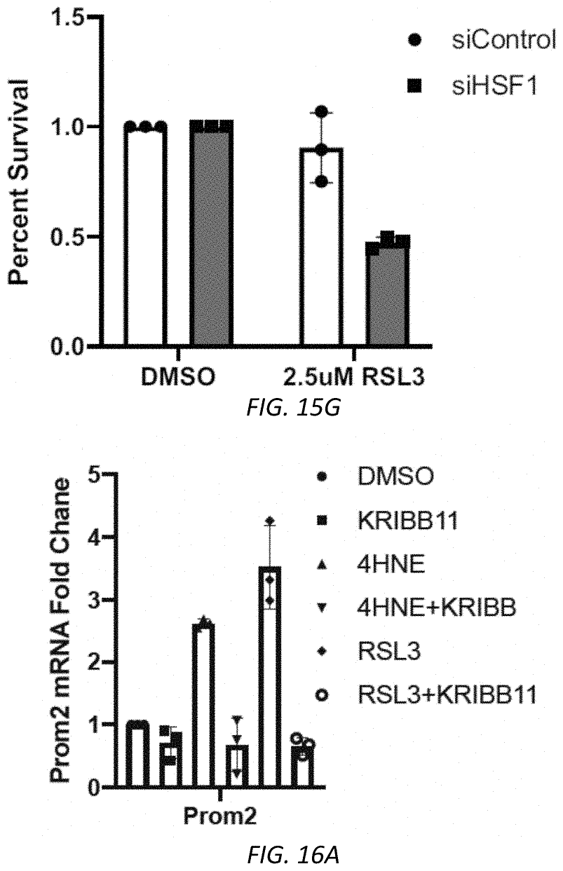

The critical issue that arose from our p38 data is how this kinase induces PROM2 mRNA expression. To address this issue, we examined ENCODE ChIP-seq data to identify transcription factors that can bind near the PROM2 promoter region. This unbiased approach yielded the heat shock factor 1 (HSF1) as the top candidate (FIG. 15A). HSF1 can induce or repress expression of genes involved in cell survival, metabolism, proliferation and immune evasion (reviewed in Dong B, 2019).

Initially, we evaluated the expression of HSF1 mRNA by qPCR in response to 4HNE or RSL3 and observed a significant increase that peaked at 15 minutes after treatment (FIG. 15B). To ascertain the role of p38 in the increase in HSF1 expression, we pre-treated MCF10A cells with the p38 inhibitor BIRB and then exposed the cells to 4HNE or RSL3. Inhibition of p38 reduced the 4HNE- or RLS3-driven increase in HSF1 to basal levels after 30 minutes of treatment (FIG. 15C). HSF1 transcriptional activity can be regulated by its expression and phosphorylation on S326 (Guettouche T, 2005). 4HNE and RSL3 increased HSF1 protein expression levels as well as phosphorylation at S326 after 60 minutes of exposure (FIG. 15D). We also observed that BIRB prevented the induction and phosphorylation of HSF1 following 4HNE or RSL3 treatment, further indicating that this increase in HSF1 activity is dependent on p38 (FIG. 15D).

Nuclear localization is required for the transcriptional function of HSF1 (K D Sarge SPM, 1993). Indeed, HSF1 expression and nuclear localization increased after 60 minutes of exposure to either 4HNE or RSL3 (FIG. 15E). Treatment with BIRB prior to 4HNE or RSLE significantly reduced the increase in expression and nuclear localization, substantiating the role of p38 in activating HSF1. These data describe a clear role for p38 in the increased expression and activation of HSF1 following a lipid peroxidation stressor.

Based on our finding that HSF1 is required for the increase in prominin2 expression, we investigated whether it is necessary for prominin2-mediated resistance to ferroptosis. MCF10A cells were transfected with either control or HSF1 targeting siRNA and assessed for their ability to increase prominin2 expression after 4HNE or RSL3 treatment. Cells depleted of HSF1 were able to increase p38 activation in response to 4HNE but not increase prominin2 expression compared to control cells (FIG. 15F). Importantly, the loss of HSF1 significantly reduced the EC50 of RSL3 required to kill MCF10A cells (FIG. 15G). Taken together, these data implicate a key and novel role for HSF1 in ferroptosis resistance by a mechanism that involves its ability to drive PROM2 transcription.

### Example 5.2. HSF1 Inhibition Sensitizes Cells to Ferroptotic Stress

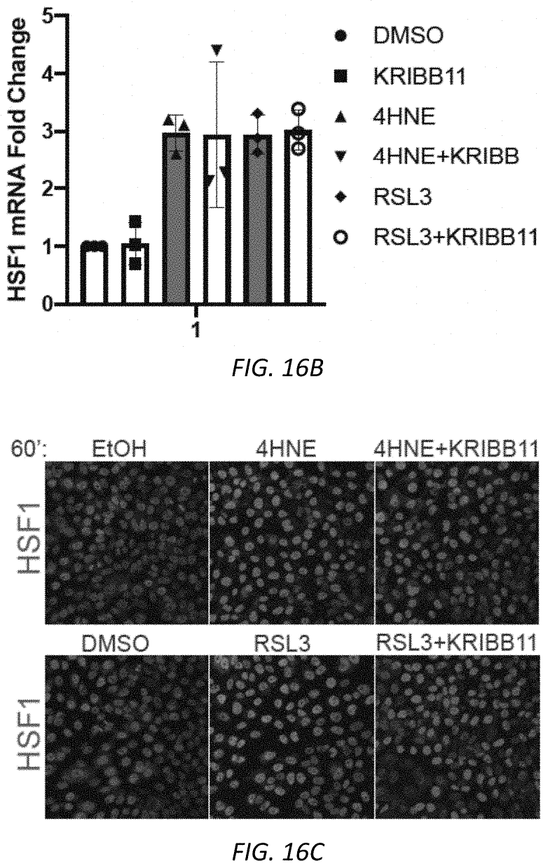

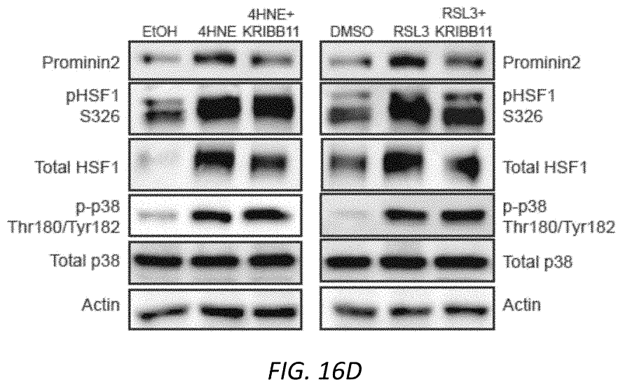

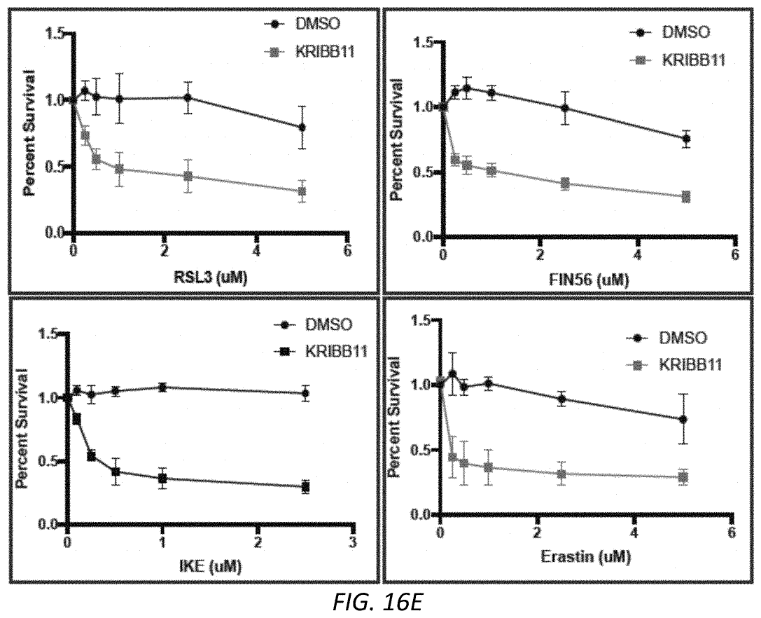

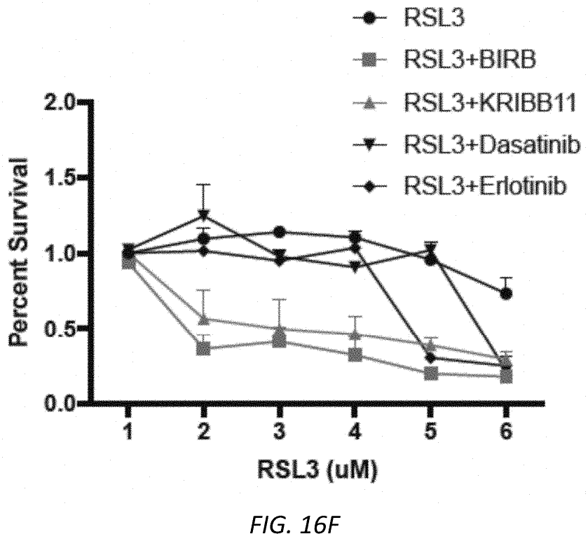

The data we obtained implicating HSF1 in ferroptosis resistance provided a potentially significant opportunity for sensitizing cells to ferroptosis because specific HSF1 inhibitors are available. Targeting HSF1 could be an effective bypass strategy as an alternative to prominin2 inhibitors. To address this possibility, we used KRIBB11, which directly targets HSF1 by inhibiting its activity as a transcription factor (Yoon Y J, 2011). Inhibition of HSF1 activity by KRIBB11 prevented the upregulation of PROM2 mRNA and prominin2 protein expression in response to either 4HNE or RSL3 (FIG. 16A, D). However, neither the expression, phosphorylation nor nuclear localization of HSF1 was affected by KRIBB11 (FIGS. 16B, C, D), consistent with its mode of action.

Having identified HSF1 as a key mediator of prominin2 expression and, consequently, the evasion of ferroptosis, we co-treated MCF10A cells with KRIBB11 and either RSL3, imidazole ketone erastin (IKE) or FIN56, compounds that induce ferroptosis through three separate mechanisms (Dixon S J, 2012; Yang W S, 2014; Zhang Y, 2019; Yang W S 2008; Shimaka K 2016). The ability of MCF10A cells to evade cell death with all three compounds was significantly when given in concert with KRIBB11 (FIG. 16E). This phenomenon was specific to ferroptosis because co-treatment with other therapeutics including dasatinib and erlotinib did not significantly improve sensitivity (FIG. 16F). These data indicate that HSF1 specifically drives a ferroptosis-resistance program and that co-treatment with a ferroptosis inducing agent and an HSF1 inhibitor can induce ferroptosis in otherwise resistant cells.

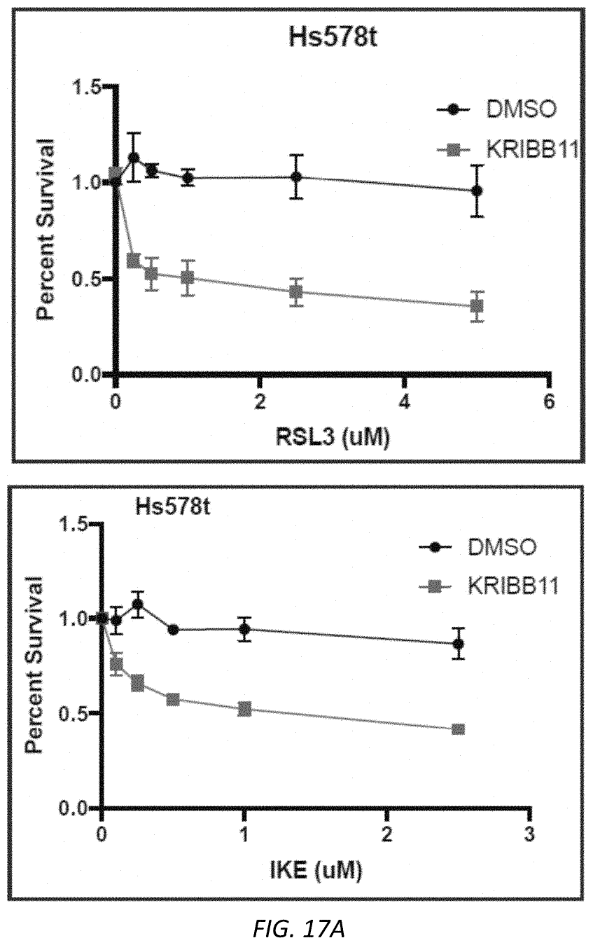

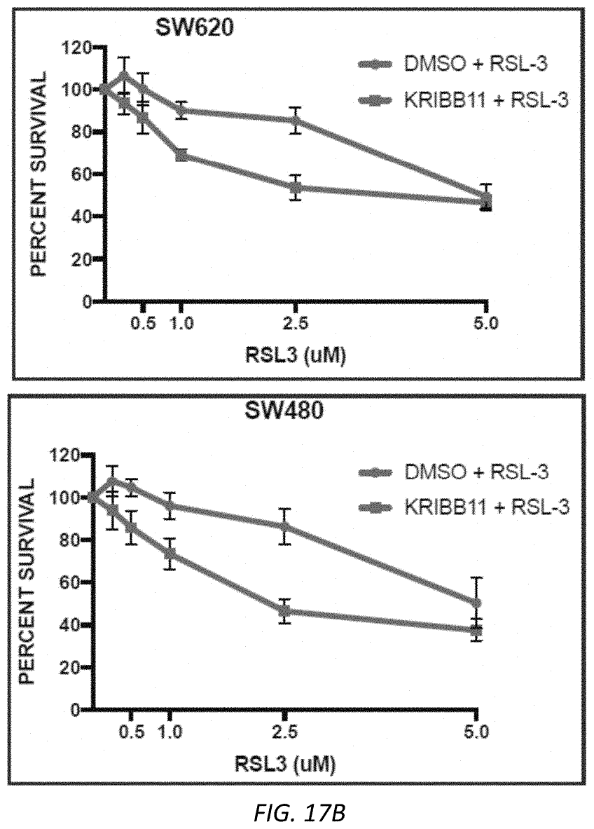

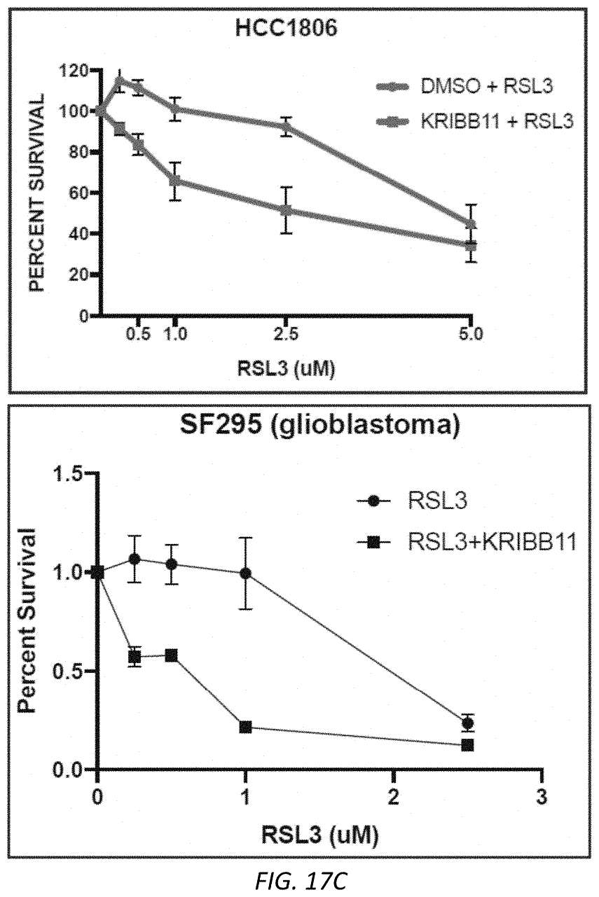

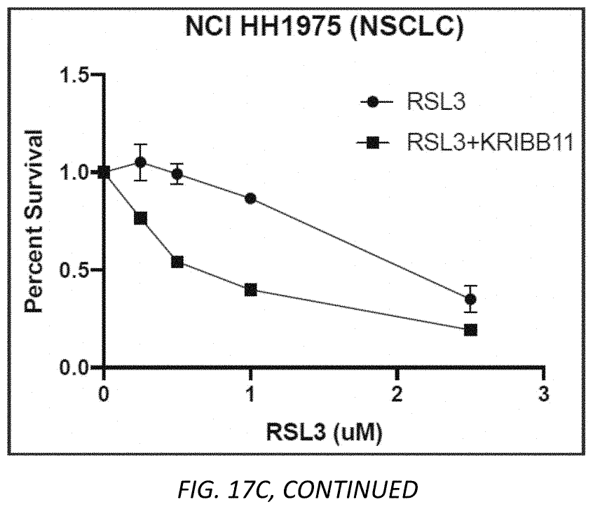

We extended our analysis of the role of HSF1 in ferroptosis resistance to transformed cells. For this purpose, we used cell lines that are resistant to ferroptosis induced by GPX4 inhibition or erastin, including the breast cancer cell lines Hs578t and HCC1806 and the colon cancer cell lines SW480 and SW620. We assessed whether KRIBB11 increased their sensitivity to either RSL3 or IKE. Indeed, the ability to resist RSL3-induced ferroptosis in all four cell lines was significantly reduced when cells were co-treated with RSL3 and KRIBB11 (FIGS. 17A, B and C).

### REFERENCES

Agmon, E., Solon, J., Bassereau, P., and Stockwell, B. R. (2018). Modeling the effects of lipid peroxidation during ferroptosis on membrane properties. Scientific reports 8, 5155.
Brown, C., Amante J J, Mercurio A M (2018). Cell clustering mediated by the adhesion protein PVRL4 is necessary for α6β4 integrin-promoted ferroptosis resistance in matrix-detached cells. The Journal of biological chemistry 293, 12741-12748.
Brown C W, A. J., Goel H L, Mercurio A M (2017). The α6β4 integrin promotes resistance to ferroptosis. The Journal of cell biology 216, 4287-4297.
Cella N, C.-U. R., Montes G S, Hynes N E, Chammas R. (1996). The lysosomal-associated membrane protein LAMP-1 is a novel differentiation marker for HC11 mouse mammary epithelial cells. Differentiation 61, 113-120.
Dixon, S.a. S., B R (2019). The Hallmarks of Ferroptosis. Annu Rev Cancer Biol 3, 35-54.
Dixon, S. J., Lemberg, K. M., Lamprecht, M. R., Skouta, R., Zaitsev, E. M., Gleason, C. E., Patel, D. N., Bauer, A. J., Cantley, A. M., Yang, W. S., et al. (2012). Ferroptosis: an iron-dependent form of nonapoptotic cell death. Cell 149, 1060-1072.
Dixon, S. J., Patel, D. N., Welsch, M., Skouta, R., Lee, E. D., Hayano, M., Thomas, A. G., Gleason, C. E., Tatonetti, N. P., Slusher, B. S., et al. (2014). Pharmacological inhibition of cystine-glutamate exchange induces endoplasmic reticulum stress and ferroptosis. eLife 3.
Do Van, B., Gouel, F., Jonneaux, A., Timmerman, K., Gelé, P., Pétrault, M., Bastide, M., Laloux, C., Moreau, C., Bordet, R., et al. (2016). Ferroptosis, a newly characterized form of cell death in Parkinson's disease that is regulated by PKC. Neurobiology of Disease 94, 169-178.
Doll S, P. B., Tyurina Y Y, Panzilius E, Kobayashi S, Ingold I, Irmler M, Beckers J, Aichler M, Walch A, Prokisch H, Trumbach D, Mao G, Qu F, Bayir H, Fullekrug J, Scheel C H, Wurst W, Schick J A, Kagan V E, Angeli J P, Conrad M. (2017). ACSL4 dictates ferroptosis sensitivity by shaping cellular lipid composition. Nature chemical biology 13, 91-98.
Dong, B., Jaeger, A. M. & Thiele, D. J. Inhibiting Heat Shock Factor 1 in Cancer: A Unique Therapeutic Opportunity. Trends Pharmacol Sci 40, 986-1005, doi:10.1016/j.tips.2019.10.008 (2019).
Fang, X., Wang, H., Han, D., Xie, E., Yang, X., Wei, J., Gu, S., Gao, F., Zhu, N., Yin, X., et al. (2019). Ferroptosis as a target for protection against cardiomyopathy. Proc Natl Acad Sci USA 116, 2672-2680.
Felder S, M. K., Moehren G, Ullrich A, Schlessinger J, Hopkins C R. (1990). Kinase activity controls the sorting of the epidermal growth factor receptor within the multivesicular body. Cell 61, 623-634.
Florek M, B. N., Janich P, Wilsch-Braeuninger M, Fargeas C A, Marzesco A M, Ehninger G, Thiele C, Huttner W B, Corbeil D (2007). Prominin-2 is a cholesterol-binding protein associated with apical and basolateral plasmalemmal protrusions in polarized epithelial cells and released into urine. Cell and tissue research 328, 31-47.
Friedmann Angeli, J. P., Schneider, M., Proneth, B., Tyurina, Y. Y., Tyurin, V. A., Hammond, V. J., Herbach, N., Aichler, M., Walch, A., Eggenhofer, E., et al. (2014). Inactivation of the ferroptosis regulator Gpx4 triggers acute renal failure in mice. Nature cell biology 16, 1180-1191.
Futter C E, P. A., Hewlett L J, Hopkins C R. (1996). Multivesicular endosomes containing internalized EGF-EGF receptor complexes mature and then fuse directly with lysosomes. The Journal of cell biology 132, 1011-1023.
Gao, M., Monian, P., Pan, Q., Zhang, W., Xiang, J., and Jiang, X. (2016). Ferroptosis is an autophagic cell death process. Cell Res 26, 1021-1032.
Geng N, S. B., Li S L, Zhong Z Y, Li Y C, Xua W L, Zhou H, Cai J H. (2018). Knockdown of ferroportin accelerates erastin-induced ferroptosis in neuroblastoma cells. Eur Rev Med Pharmacol Sci 22, 3826-3836.
Gruenberg J, M. F. (1995). Membrane transport in the endocytic pathway. Current opinion in cell biology 7, 552-563.
Guettouche, T., Boellmann, F., Lane, W. S. & Voellmy, R. Analysis of phosphorylation of human heat shock factor 1 in cells experiencing a stress. BMC Biochem 6, 4, doi:10.1186/1471-2091-6-4 (2005).
Hangauer M J, V. V., Ryan M J, Bole D, Eaton J K, Matov A, Galeas J, Dhruv H D, Berens M E, Schreiber S L, McCormick F, McManus M T. (2017). Drug-tolerant persister cancer cells are vulnerable to GPX4 inhibition. Nature 551, 247-250.
Harding C, H. J., Stahl P. (1984). Endocytosis and intracellular processing of transferrin and colloidal gold-transferrin in rat reticulocytes: demonstration of a pathway for receptor shedding. Eur J Cell Biol 35, 256-263.
Hart P D, Y. M. (1991). Ammonium chloride, an inhibitor of phagosome-lysosome fusion in macrophages, concurrently induces phagosome-endosome fusion, and opens a novel pathway: studies of a pathogenic mycobacterium and a nonpathogenic yeast. J Exp Med 174, 881-889.
Hirschhorn, T., and Stockwell, B. R. (2019). The development of the concept of ferroptosis. Free radical biology & medicine 133, 130-143.
Hou, W., Xie, Y., Song, X., Sun, X., Lotze, M. T., Zeh, H. J., 3rd, Kang, R., and Tang, D. (2016). Autophagy promotes ferroptosis by degradation of ferritin. Autophagy 12, 1425-1428.
Jászai J, F. L., Fargeas C A, Janich P, Haase M, Huttner W B, Corbeil D. (2010). Prominin-2 is a novel marker of distal tubules and collecting ducts of the human and murine kidney. Histochem Cell Biol 133, 527-539.
Jiang, L., Kon, N., Li, T., Wang, S. J., Su, T., Hibshoosh, H., Baer, R., and Gu, W. (2015). Ferroptosis as a p53-mediated activity during tumour suppression. Nature 520, 57-62.
K D Sarge, S. P. M., R I Morimoto. Activation of heat shock gene transcription by heat shock factor 1 involves oligomerization, acquisition of DNA-binding activity, and nuclear localization and can occur in the absence of stress. MCB, 1392-1407 (1993).
Kobuna, H., Inoue, T., Shibata, M., Gengyo-Ando, K., Yamamoto, A., Mitani, S., and Arai, H. (2010). Multivesicular body formation requires OSBP-related proteins and cholesterol. PLoS Genet 6.
Ma, S., Henson, E. S., Chen, Y., and Gibson, S. B. (2016). Ferroptosis is induced following siramesine and lapatinib treatment of breast cancer cells. Cell Death Dis 7, e2307.
Magtanong, L., Ko, P.-J., To, M., Cao, J. Y., Forcina, G. C., Tarangelo, A., Ward, C. C., Cho, K., Patti, G. J., Nomura, D. K., et al. (2019). Exogenous Monounsaturated Fatty Acids Promote a Ferroptosis-Resistant Cell State. Cell Chemical Biology 26, 420-432.e429.
Pan B T, T. K., Wu C, Adam M, Johnstone R M. (1985). Electron microscopic evidence for externalization of the transferrin receptor in vesicular form in sheep reticulocytes. The Journal of cell biology 101, 942-948.
Raposo, G., and Stoorvogel, W. (2013). Extracellular vesicles: exosomes, microvesicles, and friends. The Journal of cell biology 200, 373-383.
Razi M, F. C. (2006). Distinct roles for Tsg101 and Hrs in multivesicular body formation and inward vesiculation. Molecular biology of the cell 8, 3469-3483.
Saha, S. K., Islam, S. M. R., Kwak, K.-S., Rahman, M. S., and Cho, S.-G. (2019). PROM1 and PROM2 expression differentially modulates clinical prognosis of cancer: a multiomics analysis. Cancer Gene Therapy (27):147-167.
Sharma, C., and Seo, Y. H. (2018). Small Molecule Inhibitors of HSF1-Activated Pathways as Potential Next-Generation Anticancer Therapeutics. Molecules. 2018 November; 23(11): 275
Shimada K, S. R., Kaplan A, Yang W S, Hayano M, Dixon S J, Brown L M, Valenzuela C A, Wolpaw A J, Stockwell B R. (2016). Global survey of cell death mechanisms reveals metabolic regulation of ferroptosis. Nature chemical biology 12, 497-503.
Singh R D, S. A., Scheffer L, Holicky E L, Wheatley C L, Marks D L, Pagano R E. (2013). Prominin-2 expression increases protrusions, decreases caveolae and inhibits Cdc42 dependent fluid phase endocytosis. Biochemical and biophysical research communications 434, 466-472.
Stockwell, B. R., Friedmann Angeli, J. P., Bayir, H., Bush, A. I., Conrad, M., Dixon, S. J., Fulda, S., Gascón, S., Hatzios, S. K., Kagan, V. E., et al. (2017). Ferroptosis: A Regulated Cell Death Nexus Linking Metabolism, Redox Biology, and Disease. Cell 171, 273-285.
Torii, S., Shintoku, R., Kubota, C., Yaegashi, M., Torii, R., Sasaki, M., Suzuki, T., Mori, M., Yoshimoto, Y., Takeuchi, T., et al. (2016). An essential role for functional lysosomes in ferroptosis of cancer cells. The Biochemical journal 473, 769-777.
Torti S V, M. D., Paul B T, Blanchette-Farra N, Torti F M. (2018). Iron and Cancer. Annu Rev Nutr 21, 97-125.
Truman-Rosentsvit M, B. D., Spektor L, Cohen L A, Belizowsky-Moshe S, Lifshitz L, Ma J, Li W, Kesselman E, Abutbul-Ionita I, Danino D, Gutierrez L, Li H, Li K, Lou H, Regoni M, Poli M, Glaser F, Rouault T A, Meyron-Holtz E G (2018). Ferritin is secreted via two distinct non-classical vesicular pathways. Blood 131, 342-352.
Tsoi, J., Robert, L., Paraiso, K., Galvan, C., Sheu, K. M., Lay, J., Wong, D. J. L., Atefi, M., Shirazi, R., Wang, X., et al. (2018). Multi-stage Differentiation Defines Melanoma Subtypes with Differential Vulnerability to Drug-Induced Iron-Dependent Oxidative Stress. Cancer cell 33, 890-904 e895.
Viswanathan V S, R. M., Dhruv H D, Gill S, Eichhoff O M, Seashore-Ludlow B, Kaffenberger S D, Eaton J K, Shimada K, Aguirre A J, Viswanathan S R, Chattopadhyay S, Tamayo P, Yang W S, Rees M G, Chen S, Boskovic Z V, Javaid S, Huang C, Wu X, Tseng Y Y, Roider E M, Gao D, Cleary J M, Wolpin B M, Mesirov J P, Haber D A, Engelman J A, Boehm J S, Kotz J D, Hon C S, Chen Y, Hahn W C, Levesque M P, Doench J G, Berens M E, Shamji A F, Clemons P A, Stockwell B R, Schreiber S L (2017). Dependency of a therapy-resistant state of cancer cells on a lipid peroxidase pathway. Nature 547, 453-457.
Weïwer M, B. J., Lewis T A, Shimada K, Yang W S, MacPherson L, Dandapani S, Palmer M, Stockwell B R, Schreiber S L, Munoz B. (2012). Development of small-molecule probes that selectively kill cells induced to express mutant RAS. Bioorganic & medicinal chemistry letters 22, 1822-1826.
Yang, W. S., Kim, K. J., Gaschler, M. M., Patel, M., Shchepinov, M. S., and Stockwell, B. R. (2016). Peroxidation of polyunsaturated fatty acids by lipoxygenases drives ferroptosis. Proc Natl Acad Sci USA 113, E4966-4975.
Yang, W. S., SriRamaratnam, R., Welsch, M. E., Shimada, K., Skouta, R., Viswanathan, V. S., Cheah, J. H., Clemons, P. A., Shamji, A. F., Clish, C. B., et al. (2014). Regulation of ferroptotic cancer cell death by GPX4. Cell 156, 317-331.
Yang, W. S., and Stockwell, B. R. (2008). Synthetic lethal screening identifies compounds activating iron-dependent, nonapoptotic cell death in oncogenic-RAS-harboring cancer cells. Chem Biol 15, 234-245.
Yang, W. S., and Stockwell, B. R. (2016). Ferroptosis: Death by Lipid Peroxidation. Trends in cell biology 26, 165-176.
Yoon, Y. J. et al. KRIBB11 inhibits HSP70 synthesis through inhibition of heat shock factor 1 function by impairing the recruitment of positive transcription elongation factor b to the hsp70 promoter. J Biol Chem 286, 1737-1747, doi:10.1074/jbc.M110.179440 (2011).
Zhang, Y., Tan, H., Daniels, J. D., Zandkarimi, F., Liu, H., Brown, L. M., Uchida, K., O'Connor, O. A., and Stockwell, B. R. (2019). Imidazole Ketone Erastin Induces Ferroptosis and Slows Tumor Growth in a Mouse Lymphoma Model. Cell Chem Biol 26, 623-633 e629.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject who has cancer, the method comprising administering to the subject:
(i) an effective amount of an inhibitor of prominin 2 (PROM2); and/or
(ii) an effective amount of an inhibitor of heat shock factor protein 1 (HSF1) and an effective amount of one or more agents that promote ferroptosis, preferably an inhibitor of glutathione peroxidase 4 (GPX4).

2. The method of claim 1, wherein the inhibitor of PROM2 or the inhibitor of HSF1 is an inhibitory nucleic acid.

3. The method of claim 2, wherein the inhibitory nucleic acid is an antisense oligonucleotide (ASO), small interfering RNA (siRNA), or small hairpin RNA (shRNA) that binds to a PROM2 or HSF1 nucleic acid.

4. The method of claim 2, wherein the inhibitory nucleic acid comprises one or more modified bonds or bases.

5. The method of claim 4, wherein the modified bases comprise at least one nucleotide modified at the 2′ position of the sugar.

6. The method of claim 5, wherein the modified nucleotides is a 2′-O-alkyl, 2′-O-alkyl-O-alkyl or 2′-fluoro-modified nucleotide.

7. The method of claim 4, wherein the modified bonds comprise modified backbone bonds.

8. The method of claim 7, wherein the modified backbone bonds comprises phosphorothioates, phosphotriesters, methyl phosphonates, short chain alkyl or cycloalkyl intersugar linkages or short chain heteroatomic or heterocyclic intersugar linkages.

9. The method of claim 1, comprising administering the inhibitor of PROM2 and one or more agents that promote ferroptosis, preferably an inhibitor of glutathione peroxidase 4 (GPX4).

10. The method of claim 1, wherein the agent that promotes ferroptosis is selected from the group consisting of RSL3, Erastin, Imidazole ketone erastin (IKE), ML210, FIN56, Auranofin, Sulfasalazine, Artesunate, Artemisinin, Dihydroartemisinin, Sorafenib, Buthionine sulfoximine (BSO), Altretamine, Almitrine, and ML162.

11. The method of claim 1, wherein the agent that promotes ferroptosis is ascorbic acid; ultrasmall nanoparticles; elastin or carbonyl erastin analogs that inhibit GPX4; or quinazolinone inhibitors of GPX4.

12. The method of claim 1, wherein the agent that promotes ferroptosis is an inhibitory nucleic acid molecule that targets GPX4.

13. The method of claim 12, wherein the inhibitory nucleic acid is an antisense oligonucleotide (ASO), small interfering RNA (siRNA), or small hairpin RNA (shRNA) that binds to a GPX4 nucleic acid.

14. The method of claim 12, wherein the inhibitory nucleic acid comprises one or more modified bonds or bases.

15. The method of claim 14, wherein the modified bases comprise at least one nucleotide modified at the 2′ position of the sugar.

16. The method of claim 15, wherein the nucleotids is a 2′-O-alkyl, 2′-O-alkyl-O-alkyl or 2′-fluoro-modified nucleotide.

17. The method of claim 14, wherein the modified bonds comprise modified backbone bonds.

18. The method of claim 17, wherein the modified backbone bonds comprises phosphorothioates, phosphotriesters, methyl phosphonates, short chain alkyl or cycloalkyl intersugar linkages or short chain heteroatomic or heterocyclic intersugar linkages.

19. A composition comprising:
(i) an inhibitory nucleic acid targeting prominin 2 (PROM2) or an inhibitory nucleic acid targeting heat shock factor protein 1 (HSF1); and
(ii) an agent that promotes ferroptosis, preferably an inhibitor of glutathione peroxidase 4 (GPX4).

20. The composition of claim 19, wherein the agent that promotes ferroptosis is selected from the group consisting of RSL3, Erastin, Imidazole ketone erastin (IKE), ML210, FIN56, Auranofin, Sulfasalazine, Artesunate, Artemisinin, Dihydroartemisinin, Sorafenib, Buthionine sulfoximine (BSO), Altretamine, Almitrine, and ML162.

21. The composition of claim 19, wherein the agent that promotes ferroptosis is ascorbic acid; ultrasmall nanoparticles; elastin or carbonyl erastin analogs that inhibit GPX4; or quinazolinone inhibitors of GPX4.

22. The composition of claim 19, wherein the agent that promotes ferroptosis is an inhibitory nucleic acid molecule that targets GPX4.

23. The composition of claim 22, wherein the inhibitory nucleic acid is an antisense oligonucleotide (ASO), small interfering RNA (siRNA), or small hairpin RNA (shRNA) that binds to a PROM2, HSF1, or GPX4 nucleic acid.

24. The composition of claim 22, wherein the inhibitory nucleic acid comprises one or more modified bonds or bases.

25. The composition of claim 24, wherein the modified bases comprise at least one nucleotide modified at the 2′ position of the sugar.

26. The composition of claim 25, wherein the nucleotids is a 2′-O-alkyl, 2′-O-alkyl-O-alkyl or 2′-fluoro-modified nucleotide.

27. The composition of claim 24, wherein the modified bonds comprise modified backbone bonds.

28. The composition of claim 27, wherein the modified backbone bonds comprises phosphorothioates, phosphotriesters, methyl phosphonates, short chain alkyl or cycloalkyl intersugar linkages or short chain heteroatomic or heterocyclic intersugar linkages.

29. The composition of claim 19, further comprising a pharmaceutically acceptable carrier.

30. The method of claim 1, wherein the inhibitor of HSF1 is KRIBB11 n(N(2)-(1H-indazole-5-yl)-N(6)-methyl-3-nitropyridine-2,6-diamine); Quercetin; Stresgenin B; Triplotide; Cantharidin; Fisetin; Rocaglamide A; 2,4-Bis(4-hydroxy benzyl)Phenol; CL-43; celasterol; cantharidin; 2,4-Bis(4-hydroxybenzyl)phenol; minnelide; KNK437; NZ-28; PW3405; IHSF115; 4,6-Disubstituted Pyrimidine; CCT361814; α-Acyl amino carboxamides; emunin; or bisamide (CCT251236).

31. The composition of claim 19, wherein the inhibitor of HSF1 is KRIBB11 n(N(2)-(1H-indazole-5-yl)-N(6)-methyl-3-nitropyridine-2,6-diamine); Quercetin; Stresgenin B; Triplotide; Cantharidin; Fisetin; Rocaglamide A; 2,4-Bis(4-hydroxy benzyl)Phenol; CL-43; celasterol; cantharidin; 2,4-Bis(4-hydroxybenzyl)phenol; minnelide; KNK437; NZ-28; PW3405; IHSF115; 4,6-Disubstituted Pyrimidine; CCT361814; α-Acyl amino carboxamides; emunin; or bisamide (CCT251236).
